SEQUENCE LISTING (25 sequences)

SEQ ID NO: 1 - DNA, 1365 bp, Zea mays
atgagctgcagattcagagacacgatgatgtcgcgacagtgtgcaaaccgcatctttcttaccggaggttccagcttcctccatggcctcaaatctagagttgcatccagtaggagtcacggcaccgtcagactcaactggcgctgcgtgagaactaatctctggagggccgagcgccttggtgtcaaggctactccgtctgagcatatcattgcagtactgcaagcgtcagatattttcagtggcatcaggaaatggagcaagttgcaattggtcgccatgacaggggtgatggcatgtgtagttctggtagtcccttcttctgatgcagtcgatgctctcaagacatgcacttgcctgctaaaggaatgcagaatagagctggctaaatgcatagcgaacccatcctgtgcagcaaacgtagcatgcttgaacacgtgcaacaatcgacctgatgaaactgaatgtcagataaaatgcggagacctgtttgagaacagtgtagtcgatgaattcaatgattgtgctgtttcacgcaagaaatgtgttccaaaaaaatccgacgtcggcgagttccccgtccccgatccatcttcgctggttaagagcttcaacatggcagattttaatggcaaatggtacatttcaagtggcttaaacccgacgtttgacacattcgattgtcagcttcacgagtttcatgtcgagggagacaaattgatcgcgaacatcacatggagagtccgcaccccagactctggcttctttaccaggtcaaccgtgcagcgttttgtgcaggacccatcgcagcccggcatactctacaaccatgacaatgagttcctgcactatcaagatgactggtacattatctcatccaaggttgagaacaaggatgacgactacatatttgtgtactatcgtgggcgaaatgacgcatgggatggttatggtggttctgtattgtacacaagaagtaaaactgtacctgaaacaataataccggagctggaaagagctgcaaagagcgtaggtcgggacttctcgacgttcatcatgaccgacaacacctgtggtcctgagcctcctcttgtggagagaatcgagaaaactgtggtggaaggagagaagaccatcgtcagggaggtgaaggagatcgaggaggaggttgaggagctggagaaggaggaggcgtcactgtttcagaagctggcagaaggtctcatggaggtgaaacaggatttcatgaacttcttgcaggggctgagcaaggaggagatggagctgttggatcagctcaacatggaagcgactgaagttgagcaagtcttcagccgtgcactaccgttgaggaagctaaggtag

SEQ ID NO: 2 - protein, 454 aa, Zea mays
MSCRFRDTMMSRQCANRIFLTGGSSFLHGLKSRVASSRSHGTVRLNWRCVRTNLWRAERLGVKATPSEHIIAVLQASDIFSGIRKWSKLQLVAMTGVMACVVLVVPSSDAVDALKTCTCLLKECRIELAKCIANPSCAANVACLNTCNNRPDETECQIKCGDLFENSVVDEFNDCAVSRKKCVPKKSDVGEFPVPDPSSLVKSFNMADFNGKWYISSGLNPTFDTFDCQLHEFHVEGDKLIANITWRVRTPDSGFFTRSTVQRFVQDPSQPGILYNHDNEFLHYQDDWYIISSKVENKDDDYIFVYYRGRNDAWDGYGGSVLYTRSKTVPETIIPELERAAKSVGRDFSTFIMTDNTCGPEPPLVERIEKTVVEGEKTIVREVKEIEEEVEELEKEEASLFQKLAEGLMEVKQDFMNFLQGLSKEEMELLDQLNMEATEVEQVFSRALPLRKLR

SEQ ID NO: 3 - protein, 175 aa, Zea mays
DVGEFPVPDPSSLVKSFNMADFNGKWYISSGLNPTFDTFDCQLHEFHVEGDKLIANITWRVRTPDSGFFTRSTVQRFVQDPSQPGILYNHDNEFLHYQDDWYIISSKVENKDDDYIFVYYRGRNDAWDGYGGSVLYTRSKTVPETIIPELERAAKSVGRDFSTFIMTDNTCGPEP

SEQ ID NO: 4 - DNA, 1497 bp, Glycine max
atgcgatggcgactcttgtttcttcttcttcctcttccttttacatttccttattctatttctcattgtacctgcaattggcctcttctacgttgcaaggttacatttggaggcactcccaatgtacgtataaaagcaggccttacggttagaggtgatttaaggtttcacagaacagcagggtttaaatatggtcctagtgtggtagtgctcaaagttttttccaccgccaaaaggccaacacgcttgcgcttgataagaccttattgcagtttgaggggacaacacaaccaagggaaaggatattcttctctcacttctacacaaaaaccacggcaactgatgttgaaattcgaggcagatcgaatgcttgtttttctcaaagattggagtaatatacgcatcacggcagtagctgccatattggtatccattttaatgatcattccaccagctgatgctgttgatgctctcaaaacttgtgcttgcttgttgaaggattgcaggatagaactagctaagtgtctttcaaacccatcttgtgcggccaatatcgcttgtcttcaaacttgcaacaatagacctgatgaaaccgaatgccaaattaagtgtggggacctgttcgaaaatagtgtggttgatgaattcaatgagtgtgcagtctcacggaagaaatgtgtacctaagaagtctgatgtgggagaatttcctgctccaaatcctgatgtccttgttaatagctttaacattgctgatttcagtggcaagtggttcatcactagtggcttgaatcctaccttcgatacctttgactgccaattacatgaatttcacacagaatccaacaaacttgtgggtaatttgtcatggagaatacggactccagatgctggatttattactaggtctgctgagcagagatttgtacaagatccttcctatcctgggatcctttacaatcatgacaatgagtaccttcactatcaagatgactggtatattttgtcatccaagattgagaataagccagatgactacgtatttgtatactaccgaggcagaaatgatgcatgggatggttatggaggtgctgttatatacacaagaagtgcagttttgcctgaatctatagttcctgaacttgaaaaagcagctaagagtgtgggaagagactttagcacgttcatcaggacagataacacatgcgggccagagccttccctggtggaaaggctggagaaaaaggtagaggaaggagaagagaccattgtaagggaagttgaacagttagaagaagaggtggagaaggtggggaaaacggaggccaccttgtttcagaaattggcagaagggttcaaagtattccaagaagatgaggagaatttcttaagagggttatcaaaagaagaaatggagattcttgatgggctcaaaatggaagccggtgaagtagaaaagctctttggacgtgccttgccgataaggaaactaagataa

SEQ ID NO: 5 - protein, 498 aa, Glycine max
MRWRLLFLLLPLPFTFPYSISHCTCNWPLLRCKVTFGGTPNVRIKAGLTVRGDLRFHRTAGFKYGPSVVVLKVFSTAKRPTRLRLIRPYCSLRGQHNQGKGYSSLTSTQKPRQLMLKFEADRMLVFLKDWSNIRITAVAAILVSILMIIPPADAVDALKTCACLLKDCRIELAKCLSNPSCAANIACLQTCNNRPDETECQIKCGDLFENSVVDEFNECAVSRKKCVPKKSDVGEFPAPNPDVLVNSFNIADFSGKWFITSGLNPTFDTFDCQLHEFHTESNKLVGNLSWRIRTPDAGFITRSAEQRFVQDPSYPGILYNHDNEYLHYQDDWYILSSKIENKPDDYVFVYYRGRNDAWDGYGGAVIYTRSAVLPESIVPELEKAAKSVGRDFSTFIRTDNTCGPEPSLVERLEKKVEEGEETIVREVEQLEEEVEKVGKTEATLFQKLAEGFKVFQEDEENFLRGLSKEEMEILDGLKMEAGEVEKLFGRALPIRKLR

SEQ ID NO: 6 - protein, 175 aa, Glycine max
DVGEFPAPNPDVLVNSFNIADFSGKWFITSGLNPTFDTFDCQLHEFHTESNKLVGNLSWRIRTPDAGFITRSAEQRFVQDPSYPGILYNHDNEYLHYQDDWYILSSKIENKPDDYVFVYYRGRNDAWDGYGGAVIYTRSAVLPESIVPELEKAAKSVGRDFSTFIRTDNTCGPEP

SEQ ID NO: 7 - DNA, 1437 bp, Glycine max
atggcaacgtggggtgcgcactcaatgttgttatcccatggggaaggcactggcactcccaaagtatgtgtaaaagcaggccttacggttagaggtgatttaaggtttcacaggactagaggttttaaatatgctcctagtgtggtagtgctcaaagtgttggccaccgccagaaagccaacacggttgcgcttgttaagaccttattgcagtgtgagggggcaacacgaccatgggaaaggatatttttctcccactaattcacaaataccacggcaacatatattgaaattcgccgcggatcgaatgcttgcgtttctcaaagattggagtaatttacgcatcacggcagtagctggcatattggtatccgttttaatgatcattccaccagctgatgctgttgacgctctcaaaacttgtgcttgcttgttgaaggattgcaggatagaactggctaagtgtctttcaaacccatcttgtgcggccaatattgcttgtcttcaaacttgcaacaatagacctgatgagaccgaatgccaaattaaatgtggggacctgtttgaaaacagtgtggttgatgaattcaatgaatgtgcagtatcacggaagaaatgtgtacctaagaaatctgatgtgggagaatttcctgctccaaatcctgatgtccttgttaatagctttaacattgctgagttcagtggcaagtggttcatcactagtggcttaaatcctaccttcgatacctttgactgccaattacatgaatttcacacagaatccaacaaacttgtgggtaatttgtcatggagaatacggactccagatgctggatttattactaggtccgctgagcagagatttgtacaagatccttcctatcctgggatcctttacaatcatgacaataagtaccttcactatcaagatgactggtatattttgtcatccaagattgagaataaatcagatgactacgtatttgtatactaccgaggcagaaacgatgcttgggatggttatggaggtgctgttgtatacacaaggagtgcagttttgcctgaatctatagttcctgaacttgaaaaagcatctaagagtgtgggaagagactttagcaagttcatcaggacagataacacatgcgggccagagccttccctggtggaaaggctggagaaaaaggtagaggaaggagaagagaccattgtaagggaagttgaacagatagaagaagaggtggagaaggtggggaaaacggaggcgaccttatttcagaagttggcagaagggttcaaaatgtttcaagaagacgaggaaaatttcttaagagggttatcaaaagaagaaatggagattcttgatgggcttaaaatggaagccggtgaagtagaaaagctctttggacgtgccttgccgattaggaaactaagataa

SEQ ID NO: 8 - protein, 478 aa, Glycine max
MATWGAHSMLLSHGEGTGTPKVCVKAGLTVRGDLRFHRTRGFKYAPSVVVLKVLATARKPTRLRLLRPYCSVRGQHDHGKGYFSPTNSQIPRQHILKFAADRMLAFLKDWSNLRITAVAGILVSVLMIIPPADAVDALKTCACLLKDCRIELAKCLSNPSCAANIACLQTCNNRPDETECQIKCGDLFENSVVDEFNECAVSRKKCVPKKSDVGEFPAPNPDVLVNSFNIAEFSGKWFITSGLNPTFDTFDCQLHEFHTESNKLVGNLSWRIRTPDAGFITRSAEQRFVQDPSYPGILYNHDNKYLHYQDDWYILSSKIENKSDDYVFVYYRGRNDAWDGYGGAVVYTRSAVLPESIVPELEKASKSVGRDFSKFIRTDNTCGPEPSLVERLEKKVEEGEETIVREVEQIEEEVEKVGKTEATLFQKLAEGFKMFQEDEENFLRGLSKEEMEILDGLKMEAGEVEKLFGRALPIRKLR

SEQ ID NO: 9 - protein, 175 aa, Glycine max
DVGEFPAPNPDVLVNSFNIAEFSGKWFITSGLNPTFDTFDCQLHEFHTESNKLVGNLSWRIRTPDAGFITRSAEQRFVQDPSYPGILYNHDNKYLHYQDDWYILSSKIENKSDDYVFVYYRGRNDAWDGYGGAVVYTRSAVLPESIVPELEKASKSVGRDFSKFIRTDNTCGPEP

SEQ ID NO: 10 - DNA, 1065 bp, Arabidopsis thaliana
acctcaaagaaaacatcgaaatcttagagaagaagaaaagaaaaagaagagaaatgacagagaagaaagagatggaagtggtgaaagggctcaacgtggagagatacatgggccgttggtacgagattgcttctttcccatcaaggtttcagccaaagaacggcgtcgacactcgcgccacctacacccttaaccccgacggtaccatacacgtcttgaacgaaacgtggagcaacgggaagaggggttttatcgaaggcagcgcctataaggccgatcctaaaagcgacgaagccaagctcaaagtcaagttctatgtccctcctttcctcccaatcattcccgtcaccggagactactgggtgctctacatcgatcctgactaccagcacgctctcattggccagccttccaggagttatctctggatattgagcaggacggcgcaaatggaggaagagacgtataagcagctggtggagaaggcagtggaggaaggttatgacatcagcaagcttcacaagactcctcagagtgacacaccacctgagtccaacactgcccctgaagactccaagggcgtttggtggttcaaatctctcttcggcaaatagattacacggcacacaaagactttgtttcacctctttgatccttttgtctgtgtaatatactaccccctttgtctactctagttatcttccatcatctgcttttgtctatttacgacatcttcatctagttatgcaaagtcgtctcttttaccataaggaagcgcacacaaattcaatcagatcaaatctgggaaagacaattcaaatcttgcagaagttgacattatgtatttggataaacacaccacacgactcgagacttatggaatcaaaataatgcatgcaaactacagacggaattatatgtatagataagacgatagaaaaaacagtctccccccagcaaaagtttggttgactatatacccagcaggacttccagctcacagaatagtacagcatgaatttgttttcttcgcttgcttgtaaaacatctgttccaaccaataca

SEQ ID NO: 11 - protein, 186 aa, Arabidopsis thaliana
MTEKKEMEVVKGLNVERYMGRWYEIASFPSRFQPKNGVDTRATYTLNPDGTIHVLNETWSNGKRGFIEGSAYKADPKSDEAKLKVKFYVPPFLPIIPVTGDYWVLYIDPDYQHALIGQPSRSYLWILSRTAQMEEETYKQLVEKAVEEGYDISKLHKTPQSDTPPESNTAPEDSKGVWWFKSLFGK

SEQ ID NO: 12 - DNA, 1065 bp, Arabidopsis thaliana; identical to SEQ ID NO: 10

SEQ ID NO: 13 - protein, 186 aa, Arabidopsis thaliana; identical to SEQ ID NO: 11

SEQ ID NO: 14 - DNA, 1217 bp, Arabidopsis thaliana
ataaaagtctgtggcgaccaaaagaagtgagagagaaagagagagatatgatattattaagtagtagtataagcctctcaagaccagtttcttctcaaagcttctctccacctgctgccacttcaacaaggagatctcattcctctgtcacagtcaagtgctgctgttcttccagaaggttgttgaagaatcctgagttaaaatgttccttggagaatctctttgaaatccaggctttgaggaagtgttttgtttcagggtttgcagctattttgcttctctctcaggcaggccagggtatagcgttggatctctcatctggttatcagaacatttgccaactagggagtgctgctgctgtgggagaaaacaagctgactcttccatctgatggtgactcggaatcaatgatgatgatgatgatgagaggcatgactgctaagaactttgaccctgttaggtactctggaagatggtttgaagtagcttctcttaagcgtggatttgcaggtcaaggccaagaagactgtcattgcactcagggagtatacacgtttgatatgaaggaatcagccattagagtagatacattttgtgttcatggcagccctgatggatatataacaggaatcagagggaaagttcaatgcgtgggagcggaagacctcgagaaaagcgagactgacttagaaaagcaagagatgattaaagagaagtgtttcctacgatttcccaccattccttttatccccaagttgccttatgatgtcatagccacagactacgacaactacgcacttgtttctggagccaaagacaagggctttgttcaggtatactcaaggacgccaaatccaggacctgagttcatcgcaaagtacaagaactacttggcacaatttggctatgacccggaaaaaataaaggatacaccacaggactgtgaagtgactgatgctgagctagcagccatgatgtccatgccaggtatggagcaaacactgaccaaccagtttccagatcttggattaagaaagtcagtccagtttgatcccttcacaagtgtgtttgaaaccttgaagaaacttgtaccgctctatttcaaatagagcaagcttctttgctcaaattcttatgtagactataatcactgtccatatatacatatcttccagaatcaaaacactcttctgatcataacaccattgattagagaa

SEQ ID NO: 15 - protein, 353 aa, Arabidopsis thaliana
MILLSSSISLSRPVSSQSFSPPAATSTRRSHSSVTVKCCCSSRRLLKNPELKCSLENLFEIQALRKCFVSGFAAILLLSQAGQGIALDLSSGYQNICQLGSAAAVGENKLTLPSDGDSESMMMMMMRGMTAKNFDPVRYSGRWFEVASLKRGFAGQGQEDCHCTQGVYTFDMKESAIRVDTFCVHGSPDGYITGIRGKVQCVGAEDLEKSETDLEKQEMIKEKCFLRFPTIPFIPKLPYDVIATDYDNYALVSGAKDKGFVQVYSRTPNPGPEFIAKYKNYLAQFGYDPEKIKDTPQDCEVTDAELAAMMSMPGMEQTLTNQFPDLGLRKSVQFDPFTSVFETLKKLVPLYFK

SEQ ID NO: 16 - DNA, 1970 bp, Arabidopsis thaliana
aaagaaacaagaaaaaaaaaacagtctctttggcgatagcgatttggtcggagatttctccgattacattgattgggactttagtgaatcttcacagaagcaacttcacggagaacggcgagaagaatgtggaattgagagcgtcgttagaggatcgagaagcgtcggccgtttcactggagagatctagtgttgttcgttgatgaaggggttctaatttcgatacctaacggaattgatcattgataaggtctcattccggtggcaatcgtcgtcgccggaaacaatgtgcacagatacgaccttgaggggatgattcaagtgagagaaagataaagtttttttgccttcagtctgattctgcattgaatacttccagtgggctgcagcaaagcgggagatggcgaagaagagagagagtcccaagctcatcaaaatcaaagacgccattaaactcatcaaccaacaagtcagtctcattggtatcgtccttgaacaaagagaaccaaaacaatgtcgcaacaatgattggatttgtacgctacggataattgatgatacgtatccaagtcctggacttactgttaatgtatttagtaagactcttgaacaacttcctcaaatcaagaaccacgacgacatgattctgtttactcgcattaagatgcaaacttttgatagtggagaaagagtaaatgctgcttgtagtagatgggtttcttcctttgccttgttcgaaggtgtggattttgtttgttaccaatgtagtaccaattttcatgaagaagaagcattatacaagagtgccatggatgatttacgcaaagtttttgcgggttgtagccaagttattaaagctatgcagagtatatcttacagaacaaaaccttgctccgaagttttttccttcttgagagagatcaagataggaaaacgctttgatttggtgtgcaggattctccatgccgatgaagatactagcgctgtttttgtttgggacgggactgatgcacccccagcatctattctggctaagagaagtgaagaagacaaggcatttagcagtctctctgttcatactttactgtcaagagatgtactgctcagctttccaaccgttggaacgatcttgagagttcatctaagcagtcatctattttatcgggttaaacctggtgattgggtgaaactttaccatttactctgtgaagttgatagaggatcttgggtaatcaaagttaccagctctacaaaggttcatcatctcgctcaagatgatcgtttagttgaaaagatcatgaggatatatgataagagactatcatcaaagttgggacacatttcattctggtgttttccgtcacctcctggattaacagagacagatgacaattgtgccccatttgttacactgatggacatcataacattccccaaggttacatgcaagtacagatgtattgttcgggttgtagctgcatatccttggcaagtagaggatttctgttcagatgagaatcgccgtcaccaccaagttttgttgacactggaggattcaacagcgactttagaggctttcttatgcaataaagacgcagagtacttttggggtttagggtttcaggacacagagacgctgaggaagaagcggaattggttgctcggtatacgggaaagctcgaactttgtggctccaagaaatccaccatggatcgagtgctgcatcctctcctactatactaacaaagctgacccgtggaatactagattatacagaatctttggtactagactacttcattaagtatttgtaagtgttaaaagaggattaaatagtttagtattaggctcatgttaagaatgttggatttggcttacttaagtatggtagttgtgttgttttagttcatgaagaccttgtaaccaaaagaacatcttcatgatattaaccaaatccaaagactgaacac

SEQ ID NO: 17 - protein, 467 aa, Arabidopsis thaliana
MAKKRESPKLIKIKDAIKLINQQVSLIGIVLEQREPKQCRNNDWICTLRIIDDTYPSPGLTVNVFSKTLEQLPQIKNHDDMILFTRIKMQTFDSGERVNAACSRWVSSFALFEGVDFVCYQCSTNFHEEEALYKSAMDDLRKVFAGCSQVIKAMQSISYRTKPCSEVFSFLREIKIGKRFDLVCRILHADEDTSAVFVWDGTDAPPASILAKRSEEDKAFSSLSVHTLLSRDVLLSFPTVGTILRVHLSSHLFYRVKPGDWVKLYHLLCEVDRGSWVIKVTSSTKVHHLAQDDRLVEKIMRIYDKRLSSKLGHISFWCFPSPPGLTETDDNCAPFVTLMDIITFPKVTCKYRCIVRVVAAYPWQVEDFCSDENRRHHQVLLTLEDSTATLEAFLCNKDAEYFWGLGFQDTETLRKKRNWLLGIRESSNFVAPRNPPWIECCILSYYTNKADPWNTRLYRIFGTRLLH

SEQ ID NO: 18 - DNA, 1598 bp, Arabidopsis thaliana
ggcaccggccgagtcatcgcatccatcgccgttgtcgatttccttcaaacccttgcaaaaccccaacgatcagagacatggaggaggagagaagagatgactacaaattcctgagaattcaagacgctttcaaagcattgcatcttcatgttaacctcattggagtcatcgttgagctaggattctccaatggctctgattgttcatgtacgctcaaaatcgttgatccatggtattcgggatctggccttcccgttaagttcgtcgctcgtaccattcgagaccttcctcgtgttgaatcgatcggagatatcatcttactttcacgtgtaaagattgtgctgattaaccggaaaatcacagctctctgcaatgaaacaacctcttcgtcttttgctttattcaatgggaaacatagcgtggattctataccttaccagtcctcacctaaattcctgatgagagaacaggataagaacttcttatcgaatctgagagaatggatgatcacgtacaagtttgaagatgggtcatgctgtttcacatccctgaaagatatcaaagaaggcgaatgttcaaacctatcttgccagattgttcacatttccaaggtctataaagatagatggtacctctttgtttgggatggaactgagatgccaccctgcaacatcttggttaagtcagaaaggcttcctttatgtgtagaacccgagatgttgcctacatacatgttacgtaagtttcctacttttggttccgttttgaggatcatagtagatagagttagtgagaagcaggccattcactgtcttcaacctggtcaacacgtgaagcttctgaaccttttctttcaagtaaatatgggattatggaacgccacttttactccctctacaaagatgcagtacacaatgagcagagagatggaagcctttagtcctcaaagaatgtgtggggagaagttttcaccgagatggaacccaatcgcgcgttgcattagtcgttcacattctgaaatcacaggggttgcccatgacgatgctccatttgtttctctgatggacatcctgacctatcacaatgtgactgctaaattcaggtgtgttgttcggttcatacaagtgtatccccgagacgtcagaaaactccgtgatatcaacggaaatatcaaattggtagctatactagaagatgcaaccgccagaatccatgcttctctatatgctgatgaaggggaaaagtttttcgggtgtgacgaatctgatgaagaggctctagtcaagaagctgaatagattgttgggaggtgaagagatggagaaagtgccaagaaatcctccatgggtacaatgttgcttgttctcattctacaaacacaaaatggatcagtgggaaagcagaagattccgcatttttgatacttggatcaatgcttcatgagacaatcataatgctaattgtatagttattagcattaaacaaaatgtgattatttatttacatggaaccatacaataaaacattacataagaaaaatatctctctccaacgatatgtatggaagataataaagcaaaacaatctaaaataagaatc

SEQ ID NO: 19 - protein, 454 aa, Arabidopsis thaliana
MEEERRDDYKFLRIQDAFKALHLHVNLIGVIVELGFSNGSDCSCTLKIVDPWYSGSGLPVKFVARTIRDLPRVESIGDIILLSRVKIVLINRKITALCNETTSSSFALFNGKHSVDSIPYQSSPKFLMREQDKNFLSNLREWMITYKFEDGSCCFTSLKDIKEGECSNLSCQIVHISKVYKDRWYLFVWDGTEMPPCNILVKSERLPLCVEPEMLPTYMLRKFPTFGSVLRIIVDRVSEKQAIHCLQPGQHVKLLNLFFQVNMGLWNATFTPSTKMQYTMSREMEAFSPQRMCGEKFSPRWNPIARCISRSHSEITGVAHDDAPFVSLMDILTYHNVTAKFRCVVRFIQVYPRDVRKLRDINGNIKLVAILEDATARIHASLYADEGEKFFGCDESDEEALVKKLNRLLGGEEMEKVPRNPPWVQCCLFSFYKHKMDQWESRRFRIFDTWINAS

SEQ ID NO: 20 - protein, 14 aa, Artificial sequence
  Synthetic sequence
IAAIAAAIAAIAAA

SEQ ID NO: 21 - protein, 14 aa, Artificial sequence
  Synthetic sequence
IAAIQKQIAAIQKQ

SEQ ID NO: 22 - protein, 21 aa, Artificial sequence
  Synthetic sequence
IAAIAAAIAAIAAAIAAIAAA

SEQ ID NO: 23 - protein, 21 aa, Artificial sequence
  Synthetic sequence
IAAIQKQIAAIQKQIAAIQKQ

SEQ ID NO: 24 - protein, 24 aa, Artificial sequence
  Synthetic sequence
AIAAIQKQIAALQKQIAAIQKQIA

SEQ ID NO: 25 - protein, 29 aa, Artificial sequence
  Synthetic sequence
MSIEEIQKQIAAIQKQIAAIQKQIYRMTP

(19) United States
(12) Patent Application Publication
(10) Pub. No.: US 2015/0337294 A1
(43) Pub. Date: Nov. 26, 2015

(54) DESIGNER BIOACTIVE PROTEINS

(71) Applicants: Paul Skatrud, Knightstown, IN (US); Gerrit Segers, Wildwood, MO (US); Sonya Franklin, Chesterfield, MO (US); Renata Bolognesi, St. Louis, MO (US); James Roberts, Chesterfield, MO (US); Robert M. McCarroll, Lexington, MA (US); Bradon J. Fabbri, Chesterfield, MO (US); Jeffrey W. Seale, Ballwin, MO (US)

(72) Inventors: Paul Skatrud, Knightstown, IN (US); Gerrit Segers, Wildwood, MO (US); Sonya Franklin, Chesterfield, MO (US); Renata Bolognesi, St. Louis, MO (US); James Roberts, Chesterfield, MO (US); Robert M. McCarroll, Lexington, MA (US); Bradon J. Fabbri, Chesterfield, MO (US); Jeffrey W. Seale, Ballwin, MO (US)

(21) Appl. No.: 13/463,369

(22) Filed: May 3, 2012

Related U.S. Application Data
(60) Provisional application No. 61/482,566, filed on May 4, 2011.

(51) Int. Cl. C12N 15/82; A01H 5/10; C12N 5/10; C12N 15/29; A01H 5/00

(52) U.S. Cl. CPC C12N 15/1058; C12N 15/8247; C12N 15/8274; C12N 15/8273; C12N 15/8261; C12N 15/8286; C12N 15/8282; C12N 15/8283; C12N 15/8281; C12N 15/8289; C12N 15/8251

(57) ABSTRACT

Methods and compositions are provided for binding a target molecule comprising expressing a recombinant binding protein, such as an anticalin, an OB-domain protein, or an alpha-helix coiled-coil forming polypeptide in a plant. Methods for producing such a recombinant binding protein that binds to a target molecule are also provided. Further provided are plant lipocalin, OB-domain, and alpha-helix coiled-coil forming polypeptide coding sequences and recombinant polypeptides derived therefrom.

### REFERENCE TO RELATED APPLICATIONS

This application claims the benefit of U.S. provisional application No. 61/482,566 filed May 4, 2011, which is herein incorporated by reference in its entirety.

### INCORPORATION OF SEQUENCE LISTING

The sequence listing that is contained in the file named “MONS290US_seq.txt”, which is 48,378 bytes (measured in MS-WINDOWS) and created on May 3, 2012, is filed herewith by electronic submission and incorporated herein by reference

### BACKGROUND OF THE INVENTION

1. Field of the Invention

The present invention generally relates to molecular biology. More specifically, the invention relates to genes encoding polypeptides that interact with, and may bind to other molecules in planta, on the surfaces of plants and plant cells, as well as bind to molecules on and within various spaces and tissues of plant pests, and further relates to methods for modifying such genes to encode polypeptides that bind to specific target molecules.

2. Description of the Related Art

Proteins with the ability to selectively bind to target molecules by way of non-covalent interaction are important reagents in biotechnology, medicine and bioanalytics. Antibodies are a well known example of such proteins but their binding properties can be difficult to manipulate and their production is expensive. Despite the wide array of potential applications for target binding proteins, immunoglobulins remain the only widely used platforms for practical application and the use of other binding proteins, such as lectins, has been limited.

The lipocalin protein family includes small, secreted proteins that are typically characterized by a range of different molecular-recognition properties, such as their ability to specifically bind various ligands (e.g., retinoids, fatty acids and cholesterols). Lipocalins are also known to bind to specific cell-surface receptors and to facilitate the formation of macromolecular complexes. Cellular functions for lipocalins include roles in retinol transport, olfaction, pheromone signaling, and the synthesis of prostaglandins.

The OB-domain family of proteins shares some of the structural features of the lipocalin proteins, such a stabilized beta-barrel structure (Murzin, 1993) Likewise, OB-domain proteins display high affinity binding to ligands such as polynucleotides and are involved in a variety of DNA-binding activities such as telomere maintenance.

Certain single-chain, triple-stranded coiled-coil forming polypeptides can bind with high affinity to a wide range of molecular targets. These polypeptides exhibit an alpha helical configuration and are 10 to 15 times smaller than antibodies with a molecular weight of between 10 and 14 kDa. The structural characteristics allow these alpha-helical coiled-coil forming polypeptides to bind to certain targets that are not easily accessible to antibodies and can present multi-specific target binding by displaying more than one antigen binding site.

Recently, members of the mammalian lipocalin family, certain OB-domain proteins, alpha-helical coiled-coil forming polypeptides, and other synthetic polypeptides have become subjects of research concerning proteins having defined ligand-binding properties.

### SUMMARY OF THE INVENTION

In a first aspect, the invention provides a method for binding a target molecule comprising expressing a recombinant beta-barrel binding protein, or a polypeptide which forms an alpha-helical coiled-coil structure, in a plant and contacting the target molecule with the recombinant plant beta-barrel binding, or coiled-coil-forming polypeptide. In one embodiment the recombinant beta-barrel binding protein is a recombinant plant anticalin. In certain embodiments the recombinant plant anticalin has a mass of between about 15 and 25 KDa; wherein the recombinant plant anticalin comprises an amino acid sequence at least 80% identical to an anticalin domain selected from the group consisting of SEQ ID NOs: 3, 6 and 9; or wherein the recombinant plant anticalin forms a beta-barrel structure and the loops of the beta-barrel are modified relative to a native plant anticalin. In other embodiments, the recombinant plant beta-barrel binding protein is a recombinant plant OB-fold protein. In particular embodiments the recombinant plant OB-fold protein has a mass of between about 10 and 25 KDa; wherein the recombinant plant OB-fold protein comprises an amino acid sequence at least 80% identical to an OB-fold domain selected from the group consisting of SEQ ID NOs: 17 or 19; or wherein the recombinant plant OB-fold protein forms a beta-barrel structure and the amino acid sequence of a beta-strand in an OB-fold domain binding face or an OB-fold domain loop of the beta-barrel are modified relative to a native plant OB-fold protein.

In other embodiments, the polypeptide which forms an alpha-helical coiled-coil structure comprises a sequence selected from the group consisting of SEQ ID NOs:20-25. In certain embodiments the expressed polypeptide further comprises a localization sequence. In other embodiments the polypeptide which forms an alpha-helical coiled-coil structure comprises 1-3 ligand binding domains.

The invention also provides a method wherein contacting the target molecule with the recombinant plant beta-barrel binding protein or the polypeptide which forms an alpha-helical coiled-coil structure comprises contacting the plant with the target molecule; or wherein contacting the target molecule with the recombinant plant beta-barrel binding protein or polypeptide which forms an alpha-helical coiled-coil structure comprises providing the recombinant plant beta-barrel binding protein or polypeptide which forms an alpha-helical coiled-coil structure in the diet of an insect pest wherein the insect pest comprises the target molecule. In certain embodiments the target molecule is present in cells of the midgut of the insect pest.

In yet other embodiments of the method of the present invention, the recombinant plant beta-barrel binding protein or polypeptide which forms an alpha-helical coiled-coil structure is produced by a method comprising: (a) producing a plurality of recombinant coding sequence for a plant beta-barrel binding protein or for a polypeptide which forms an alpha-helical coiled-coil structure to generate a library of recombinant protein sequences; and (b) screening the library of recombinant sequences to identify a recombinant plant beta-barrel binding protein sequence or a polypeptide which forms an alpha-helical coiled-coil structure that binds to a target molecule; or wherein the method comprises: (a) producing a plurality of recombinant coding sequence for a plant beta-barrel binding protein or for a polypeptide which forms an alpha-helical coiled-coil structure to generate a library of recombinant plant beta-barrel binding protein sequences or sequences which encode a polypeptide which forms an alpha-helical coiled-coil structure; (b) screening the library of recombinant plant beta-barrel binding protein sequences or sequences which encode a polypeptide which forms an alpha-helical coiled-coil structure to identify a recombinant polypeptide sequence that binds to a target molecule; (c) producing a further plurality of recombinant coding sequences for the plant beta-barrel binding protein or a polypeptide which forms an alpha-helical coiled-coil structure to generate a further library of coding sequences; and (d) screening the further library of coding sequences to identify a plant beta-barrel binding protein sequence, or a sequence of a polypeptide which forms an alpha-helical coiled-coil structure, with enhanced binding to a target molecule; or wherein the method further comprises, after steps (a)-(d): (e) repeating steps (c)-(d) one or more times to generate a recombinant plant beta-barrel binding protein, or a polypeptide which forms an alpha-helical coiled-coil structure, with further enhanced binding to a target molecule.

In some embodiments screening the library comprises the use of phage display or bacterial anchored periplasmic expression to identify binding of a recombinant plant beta-barrel binding protein, or a sequence of a polypeptide which forms an alpha-helical coiled-coil structure, to a target molecule; or wherein the binding of a recombinant plant beta-barrel binding protein, or a polypeptide which forms an alpha-helical coiled-coil structure, to a target molecule is identified by fluorescence-activated cell sorting or magnetic separation.

Alternatively, in some embodiments of the method, the recombinant plant beta-barrel binding protein or the polypeptide which forms an alpha-helical coiled-coil structure, is produced by a method comprising: (a) producing a plurality of recombinant plant beta-barrel binding proteins or polypeptides which form an alpha-helical coiled-coil structure; and (b) screening the recombinant plant beta-barrel binding proteins, or polypeptides which form an alpha-helical coiled-coil structure, to identify a recombinant plant beta-barrel binding protein, or a polypeptide which forms an alpha-helical coiled-coil structure, that binds to a target molecule. In particular embodiments, identifying a recombinant plant beta-barrel binding protein, or a polypeptide which forms an alpha-helical coiled-coil structure, that binds to a target molecule comprises determining the sequence of the recombinant plant beta-barrel binding protein or determining the sequence of a polypeptide which forms an alpha-helical coiled-coil structure; optionally wherein the sequence is determined by mass spectroscopy.

In some embodiments, the target molecule is a small molecule, a protein, or a carbohydrate. In particular embodiments the small molecule target is an ACCase inhibitor, ALS inhibitor, a triazine, a bypyridilium, an auxin, glycine or a triketone; wherein the protein target molecule is a protein expressed in the plant; wherein the protein target molecule is a peritrophin, chitinase, aminopeptidase, cystein protease, trypsin, chymotrypsin or carboxypeptidase; wherein the carbohydrate target molecule is chitin; wherein the target molecule is present in the gut of an insect pest; or wherein the binding of the target molecule occurs in the plant.

The invention also provides a method wherein expressing the recombinant plant beta-barrel binding protein or the polypeptide which forms an alpha-helical coiled-coil structure in the plant confers a trait of agronomic interest to the plant. In particular embodiments the trait of agronomic interest is selected from the group consisting of herbicide tolerance, drought resistance, insect resistance, fungus resistance, virus resistance, bacteria resistance, male sterility, cold tolerance, salt tolerance, increased yield, enhanced oil composition, increased oil content, enhanced nutrient use efficiency and altered amino acid content.

In some embodiments of the method of the present invention, the recombinant plant beta-barrel binding protein or the polypeptide which forms an alpha-helical coiled-coil structure binds to at least two different target molecules; or wherein the recombinant plant beta-barrel binding protein coding sequence or the sequence encoding the polypeptide which forms an alpha-helical coiled-coil structure is fused to a second coding sequence encoding a polypeptide of agronomic interest. In some embodiments the second coding sequence encodes a recombinant plant beta-barrel binding protein or encodes a polypeptide which forms an alpha-helical coiled-coil structure; or wherein the second coding sequence encodes a pore-forming toxin.

In certain embodiments, the plant in which the recombinant beta-barrel binding protein, or the polypeptide which forms an alpha-helical coiled-coil structure, is expressed is selected from the group consisting of wheat, maize, rye, rice, corn, oat, barley, turfgrass, sorghum, millet, sugarcane, tobacco, tomato, potato, soybean, cotton, canola, sunflower and alfalfa.

Another aspect of the invention comprises a polynucleotide molecule comprising a nucleic acid with a sequence selected from the group consisting of: (a) a nucleic acid sequence encoding a polypeptide comprising a sequence at least 80% identical to SEQ ID NOs: 2, 3, 5, 6, 8 or 9; (b) a nucleic acid sequence at least 80% identical to SEQ ID NOs: 1, 4, or 7; (c) a nucleic acid sequence encoding a polypeptide comprising the sequence of SEQ ID NOs: 2, 3, 5, 6, 8 or 9; (d) a nucleic acid sequence of SEQ ID NOs: 1, 4 or 7; and (e) a nucleic acid sequence encoding a polypeptide comprising the sequence selected from the group consisting of SEQ ID NOs:20-25. In some embodiments the polynucleotide molecule is operably linked to a heterologous promoter functional in a plant cell.

A transgenic plant or plant part, such as a plant cell, transformed with such a polynucleotide molecule is another aspect of the invention. The transgenic plant may be selected from the group consisting of wheat, maize, rye, rice, corn, oat, barley, turfgrass, sorghum, millet, sugarcane, tobacco, tomato, potato, soybean, cotton, canola, sunflower and alfalfa. A transgenic plant cell transformed with the polynucleotide molecule is a further embodiment of the invention. A seed of the transgenic plant, comprising the nucleic acid, is another embodiment of the invention.

The invention further provides a method wherein enhanced binding to a target molecule comprises enhanced binding specificity or affinity to a target molecule. Thus, the invention also provides a method wherein the plurality of recombinant coding sequence for a plant beta-barrel binding protein or alpha-helical coiled-coil polypeptide comprise beta-barrel binding protein coding sequences or alpha-helical coiled-coil polypeptide coding sequences that are mutated by random mutagenesis; wherein the plurality of recombinant coding sequence for a plant beta-barrel binding protein or alpha-helical coiled-coil polypeptide comprise beta-barrel binding protein coding sequence or alpha-helical coiled-coil polypeptide coding sequence that are mutated by site-directed mutagenesis; wherein the plurality of recombinant coding sequence for a plant beta-barrel binding protein or alpha-helical coiled-coil polypeptide comprise beta-barrel binding protein coding sequences or alpha-helical coiled-coil polypeptide coding sequences comprising an amino acid deletion or substitution; wherein 2 or more plant beta-barrel binding protein sequences or alpha-helical coiled-coil polypeptide sequences are modified to generate the library; wherein the recombinant beta-barrel binding protein is a recombinant plant anticalin; wherein the recombinant beta-barrel binding protein is a recombinant plant OB-domain protein; wherein screening the library comprises the use of phage display or bacterial anchored periplasmic expression to identify binding of a plant anticalin to a target molecule; wherein the binding of a plant anticalin to a target molecule is identified by fluorescence-activated cell sorting or magnetic separation; or wherein the target molecule is a small molecule, a protein or a carbohydrate.

In certain embodiments the plurality of plant beta-barrel binding protein coding sequences is produced by modification of an anticalin coding sequence selected from the group consisting of a polynucleotide molecule encoding a polypeptide comprising any of SEQ ID NOs: 2, 3, 5, 6, 8, and 9; or wherein the plurality of plant beta-barrel binding protein coding sequences is produced by modification of an anticalin coding sequence selected from the group consisting of a polynucleotide molecule encoding a polypeptide comprising any of SEQ ID NOs: 3, 6, and 9. In other embodiments, the plurality of plant beta-barrel binding protein coding sequences is produced by modification of a plant OB-domain coding sequence selected from the group consisting of a polynucleotide molecule encoding a polypeptide comprising SEQ ID NO: 17 or SEQ ID NO:19. Further, in particular embodiments the small molecule target molecule is an ACCase inhibitor, an ALS inhibitor, a triazine, a bypyridilium, an auxin, glycine or a triketone; wherein the protein target molecule is a peritrophin, chitinase, aminopeptidase, cystein protease, trypsin, chymotrypsin or carboxypeptidase; or wherein the carbohydrate target molecule is chitin.

As used herein the terms “encode” or “encoding” with reference to a nucleic acid are used to make the invention readily understandable by the skilled artisan however these terms may be used interchangeably with “comprise” or “comprising” respectively.

As used herein the specification, “a” or “an” may mean one or more. As used herein in the claim(s), when used in conjunction with the word “comprising”, the words “a” or “an” may mean one or more than one.

The use of the term “or” in the claims is used to mean “and/or” unless explicitly indicated to refer to alternatives only or the alternatives are mutually exclusive, although the disclosure supports a definition that refers to only alternatives and “and/or.” As used herein “another” may mean at least a second or more.

Throughout this application, the term “about” is used to indicate that a value includes the inherent variation of error for the device, the method being employed to determine the value, or the variation that exists among the study subjects.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the invention. The invention may be better understood by reference to one or more of these drawings in combination with the detailed description of specific embodiments presented herein:

FIG. 1: A schematic representation of a method for producing a recombinant plant anticalin with binding affinity for a target molecule.

FIG. 2: A schematic representation of a method for producing a recombinant plant OB-domain protein with binding affinity for a target molecule.

FIG. 3: A schematic representation of a method for producing a recombinant alpha-helical coiled-coil alphabody protein with binding affinity for a target molecule.

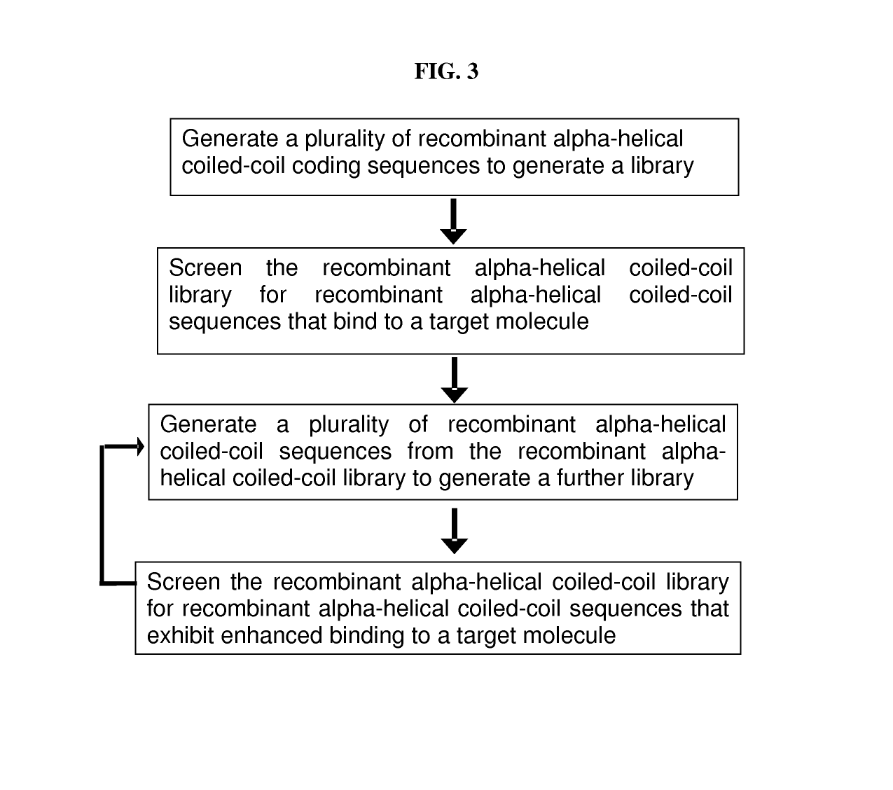

### BRIEF DESCRIPTION OF THE SEQUENCES

SEQ ID NOs:1-2 Nucleotide and amino acid sequence for Z. mays violaxanthin de-epoxidase. The lipocalin domain corresponds to amino acids 188-362.

SEQ ID NO:3 Amino acid sequence for the lipocalin domain of Z. mays violaxanthin de-epoxidase of SEQ ID NO: 2.

SEQ ID NOs:4-5 Nucleotide and amino acid sequence for G. max violaxanthin de-epoxidase. The lipocalin domain corresponds to amino acids 232-406.

SEQ ID NO:6 Amino acid sequence for the lipocalin domain of G. max violaxanthin de-epoxidase of SEQ ID NO: 5.

SEQ ID NOs:7-8 Nucleotide and amino acid sequence for G. max violaxanthin de-epoxidase. The lipocalin domain corresponds to amino acids 212-386.

SEQ ID NO:9 Amino acid sequence for the lipocalin domain of G. max violaxanthin de-epoxidase of SEQ ID NO: 8.

SEQ ID NOs:10-11 Nucleotide and amino acid sequence for Z. mays temperature-induced lipocalin-1.

SEQ ID NOs:12-13 Nucleotide and amino acid sequence for Z. mays temperature-induced lipocalin-2.

SEQ ID NOs:14-15 Nucleotide and amino acid sequence for Z. mays chloroplastic lipocalin.

SEQ ID NOs: 16-17 Nucleotide and amino acid sequence for A. thaliana POT1a (NCBI Accession No. NM_{—}001124799.1; see, e.g., Shakirov et al., 2005).

SEQ ID NOs: 18-19 Nucleotide and amino acid sequence for A. thaliana POT1b (NCBI Accession No. NM_{—}120714.3; see, e.g., Shakirov et al., 2005).

SEQ ID NOs:20-25 Representative synthetic polypeptide sequences that form alpha helical coiled-coil structures (e.g. see U.S. Patent Applic. Publ. 20100305304).

### DETAILED DESCRIPTION OF THE INVENTION

The invention overcomes limitations of the prior art by providing novel methods and compositions for binding target molecules by expressing an engineered protein, such as a recombinant beta-barrel binding protein, or a polypeptide that can assume an alpha helix configuration in a plant.

Coding sequences that encode such binding proteins can be altered to encode recombinant binding proteins with the ability to bind virtually any target molecule. Expression of such recombinant coding sequences in plants can therefore be used to confer a variety of agronomically important traits to a plant.

The invention also provides plant coding sequences for lipocalins, for plant OB-domain coding sequences, and for polypeptides that can assume an alpha helix configuration in a plant, that can serve as the basis for modification to generate recombinant binding proteins. Because the original lipocalin and OB-domain polypeptides are naturally expressed in plants, resultant recombinant proteins are more likely to be expressed efficiently and should not result in allergic reactions in animals consuming plants that express the sequences. Further, the small size (e.g. molecular weight of 10-14 kDa, one-tenth the size of typical antibodies), and simple structure of alpha helix-forming polypeptides should also allow their efficient in planta expression and in vivo function. Because such polypeptides may retain their binding properties while tolerating various amino acid substitutions, they offer an alternative to much larger immunoglobulin molecules as proteinaceous scaffolds and for affecting the function of target molecules.

Recombinant plant beta-barrel binding proteins find use in a wide array of applications. For example, recombinant beta-barrel binding proteins can be used to direct a fused second polypeptide, such as a toxin (e.g., a Cry protein), to a target molecule such as molecule found on the surface of an insect cell. A plant expressing such a fusion protein would thereby provide a directed toxin that when ingested by an insect pests would result in morbidity or mortality of the insect, thereby protecting the plant from insect damage. In other aspects, a beta-barrel binding protein may bind-to and alter the function of a molecule within a plant such as a hormone. Plant beta-barrel binding proteins can also be used to sequester bound molecules or to alter their distribution. For instance, an herbicide binding protein may be used to sequester a herbicide and increase herbicide tolerance in a plant.

Recombinant plant beta-barrel binding protein molecules may also bind to two or more different target molecules. For example, a recombinant protein may comprise two binding pockets that allow for binding to two different target molecules. Alternatively or additionally, a beta-barrel binding protein may be provided as a fusion protein with a second target binding molecule (e.g., an anticalin) to form a dual binding protein. Such dual-binding proteins can be used to facilitate co-localization of bound target molecules. For instance, an herbicide-binding anticalin may also bind to an enzyme that breaks down an herbicide thereby targeting the herbicide for efficient degradation. In another example, a beta-barrel binding protein may comprise a domain that binds double-stranded RNA and a second domain that binds to a target on an insect, fungal or bacterial pest. In this case, the anticalin or OB-domain protein could increase the efficacy of a double-stranded RNA directed to a pest by specifically targeting the RNA to the pest of interest.

The lipocalins encoded by various species share a low level of overall sequence conservation, often with sequence identities of less than 20%. Despite this an array of plant lipocalin sequences are provided herein and can be used as the basis for generating recombinant plant anticalin polypeptides. Lipocalin polypeptides maintain a highly conserved folding pattern which provides a scaffold for alteration of the coding sequence (e.g., amino acid deletions, insertions or substitutions). The central part of the lipocalin structure forms a single eight-stranded anti-parallel beta-sheet closed back on itself to form a continuously hydrogen-bonded beta-barrel structure. One end of the barrel is sterically blocked by the N-terminal peptide segment that runs across its bottom as well as three peptide loops connecting the beta-strands. The opposing end of the beta-barrel is open to the solvent and encompasses a target molecule-binding site. The four amino acid loops at this end of the beta-barrel are examples of regions in which amino acid changes can be introduced to alter the binding properties of the polypeptide (see, e.g., U.S. Pat. No. 7,250,297 and U.S. Appln. No. 20090325875, each of which is incorporated by reference herein). In certain cases, the interior of the beta-barrel structure can serve as part of the ligand binding domain or may form a binding pocket for a second ligand.

Accordingly, amino acid positions on the interior of the beta-barrel are also sites where amino acid modifications can be incorporated. Thus, by altering the amino acids sequence of the lipocalin protein, such as in the four beta-barrel loops and/or the residues on the interior of the beta barrel, the protein can be made to accommodate binding of molecular targets of different size, shape, and chemical character.

Certain residues of polypeptides termed “Alphabodies™” have been synthesized and studied for their binding properties (e.g. US Patent Applic. Publ. 2010/0305304; WO 2009/030780; WO2010/066740; Complix NV; Hasselt, Belgium). These polypeptides may non-covalently interact and form thermodynamically stable parallel or antiparallel alpha-helical coiled-coil scaffolds. Recombinant alpha-helix coiled-coil forming polypeptides, i.e. proteinaceous scaffold molecules which can self assemble and non-covalently interact to form stable alpha helical coiled-coil structures, may find use in a wide array of applications. The binding domain of the alpha-helix coiled-coil forming polypeptides may comprise one or more peptides, for instance one, two (“dimer”), or three (“trimer”) peptides. For example, recombinant alpha-helix coiled-coil forming polypeptides can be fused with one or more other functional domains and/or localization sequences and used to create synthetic molecules with novel binding activities and functions. Additional coding sequences to effect desired molecular interactions may also be provided by engineering a gene having codons specifying linker sequences adjacent to alpha-helix coiled-coil forming polypeptide heptad repeat sequences (e.g. see SEQ ID NOs:20-25). Such coding sequences may be expressed in planta or for instance in an expression system such as a microbial or other cell culture, followed by the contacting of expressed protein with a plant or plant part, or with a cell of a plant pest or plant pathogen. If expressed in a cell culture, purification and formulation of the protein may be performed, for instance to enhance its stability, absorption, and/or efficacy.

Thus, expression of an engineered alpha-helix coiled-coil forming polypeptide that has high specificity and affinity for a molecular target may allow for the killing of, or prevention of growth, replication, differentiation, or pathogenesis of, an organism considered to cause disease, be a pest, or alternatively intrinsically affect the growth and yield of a plant. A binding target for an alpha-helix coiled-coil forming polypeptide may be on a cell surface, or intracellular such as in an organelle, and binding may occur following in planta expression of (a) recombinant gene(s) encoding one or more alpha-helix coiled-coil forming polypeptide(s). Alternatively, the alpha-helix coiled-coil forming polypeptide may be expressed in a cell culture, such as a bacterial cell culture, for subsequent application to a plant. Thus, the alpha-helix coiled-coil forming polypeptide may contact its target following its topical application to a plant.

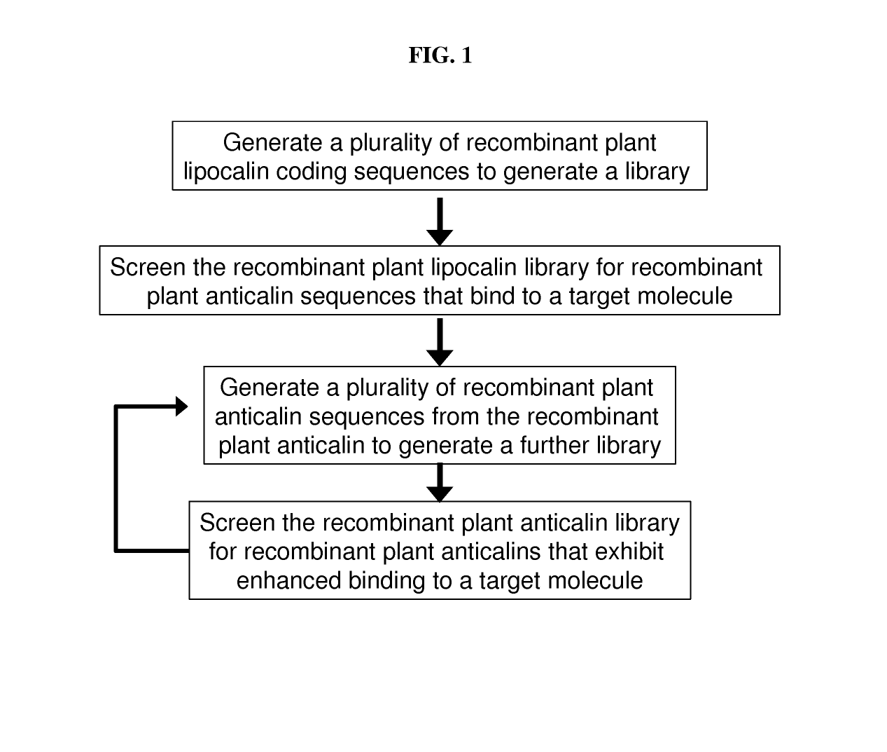

Accordingly, the invention provides methods for producing recombinant molecules, such as plant anticalins that bind to a target molecule of interest, by altering the coding sequence of a plant lipocalin, or by otherwise effecting expression of a beta-barrel binding protein or an alpha-helix coiled-coil forming polypeptide. An exemplary protocol for such a method is provided as FIG. 1. For instance, a starting plant lipocalin sequence can be used as the basis to make a plurality of recombinant sequences. Such recombinant sequences can be by made by random mutagenesis or by incorporating specific changes in the protein (e.g., in polypeptide loops at the end of the beta-barrel or at amino acid position that form the interior of the beta-barrel) and may comprise amino acid insertions, deletions or substitutions. The library of the recombinant lipocalin sequences is then screened for binding to a target molecule to identify recombinant anticalin polypeptides that bind to the molecule. In certain aspects, a recombinant plant anticalin sequence that binds to the target molecule can be subjected to alteration to a generate a further library of the sequences that can be screened for enhanced binding to the target molecule and/or binding or reduced binding to a second molecule. By subjecting an anticalin to iterative alteration of the coding sequence and screening (or selection), anticalin sequences can be produced that have high affinity and/or high specificity for a target molecule or set of target molecules.

### I. MODIFICATION OF BETA-BARREL BINDING PROTEIN CODING SEQUENCES

In certain aspects, the coding sequence for plant lipocalin polypeptides are altered, for example, to produce libraries of recombinant lipocalin coding sequences and OB-domain proteins that bind to a target molecule. In certain aspects the entire coding region for a plant lipocalin may be used, however in other aspects, only sequences from the lipocalin domain are used for such modification. For example, SEQ ID NOs: 2, 5 and 5 provide the coding sequences for three plant lipocalin proteins and the lipocalin domains for each are identified in SEQ ID NOs: 3, 6 and 9, respectively. Likewise, additional plant lipocalin coding sequences are provided in Table 1 and the lipocalin domain of each can be readily identified by comparison to the lipocalin domains of SEQ ID NOs: 3, 6 and 9.

TABLE 1
Additional plant lipocalin coding sequences

| Organism | Query_ID | Hit_ID |
|---|---|---|
| Arabidopsis | gi\|77744847\|gb\|ABB02377.1\| | AT5G58070.1 |
| Arabidopsis | gi\|195657393\|gb\|ACG48164.1\| | AT5G58070.1 |
| Arabidopsis | gi\|194706588\|gb\|ACF87378.1\| | AT3G47860.1 |
| Maize_B73 | gi\|77744847\|gb\|ABB02377.1\| | Zm_B73_UN.G54.19.pep |
| Maize_B73 | gi\|195657393\|gb\|ACG48164.1\| | Zm_B73_UN.G7537.11.pep |
| Maize_B73 | gi\|194706588\|gb\|ACF87378.1\| | Zm_B73_CR02.G135120.1.pep |
| Medic | gi\|77744847\|gb\|ABB02377.1\| | Medtr8g146020.1 |
| Medic | gi\|195657393\|gb\|ACG48164.1\| | Medtr8g146020.1 |
| Medic | gi\|194706588\|gb\|ACF87378.1\| | Medtr5g099410.1 |
| Rice | gi\|77744847\|gb\|ABB02377.1\| | LOC_Os02g39930.1#13102.m04435#protein |
| Rice | gi\|195657393\|gb\|ACG48164.1\| | LOC_Os08g34150.1#13108.m03572#protein |
| Rice | gi\|194706588\|gb\|ACF87378.1\| | LOC_Os04g53490.1#13104.m05507#protein |
| Sorghum | gi\|77744847\|gb\|ABB02377.1\| | Sb04g025860.1 |
| Sorghum | gi\|195657393\|gb\|ACG48164.1\| | Sb07g021580.1 |
| Sorghum | gi\|194706588\|gb\|ACF87378.1\| | Sb06g029210.1 |
| Soybean_Williams82 | gi\|77744847\|gb\|ABB02377.1\| | Gm_W82_CR17.G26790.1.pep |
| Soybean_Williams82 | gi\|195657393\|gb\|ACG48164.1\| | Gm_W82_CR13.G172200.1.pep |
| Soybean_Williams82 | gi\|194706588\|gb\|ACF87378.1\| | Gm_W82_CR14.G10830.1.pep |

Recombinant beta-barrel binding protein coding regions can retain substantial sequence similarity to the plant lipocalin, lipocalin domain, or OB-domain from which it was derived. Thus, in certain aspects, a recombinant plant beta-barrel binding protein is 75%, 80%, 85%, 90%, 95%, 98%, or 99% identical to SEQ ID NOs: 2, 3, 5, 6, 8, 9, 17, 19 or one of the sequences provided in Table 1. Accordingly, the nucleic acid sequence encoding an anticalin may bind with high stringency to the complement of a nucleic acid sequence encoding a plant lipocalin domain (e.g., SEQ ID NOs: 1, 4, 7 or one of the polynucleotides provided in Table 1).

As used herein, “hybridization” or “hybridizes” is understood to mean the forming of a double or triple stranded molecule or a molecule with partial double or triple stranded nature. As used herein “stringent condition(s)” or “high stringency” are those conditions that allow hybridization between or within one or more nucleic acid strand(s) containing complementary sequence(s), but precludes hybridization of random sequences. Stringent conditions tolerate little mismatch between a nucleic acid and a target strand. Such conditions are well known to those of ordinary skill in the art, and may be used for applications requiring high selectivity. Medium stringent conditions may comprise relatively low salt and/or relatively high temperature conditions, such as provided by about 1×SSC, and 65° C. High stringency may be defined as 0.02M to 0.10M NaCl and 50° C. to 70° C. Specific examples of such conditions include 0.02M NaCl and 50° C.; 0.02M NaCl and 60° C.; and 0.02M NaCl and 70° C.

Alterations of the native amino acid sequence to produce variant polypeptides, such as recombinant lipocalins, can be prepared by a variety of means known to those ordinarily skilled in the art. For instance, amino acid substitutions can be conveniently introduced into the polypeptides by changing the sequence of the nucleic acid molecule at the time of synthesis. Site-specific mutations can also be introduced by ligating into an expression vector a synthesized oligonucleotide comprising the modified sequence. Alternately, oligonucleotide-directed, site-specific mutagenesis procedures can be used, such as disclosed in Walder et al. (1986); and U.S. Pat. Nos. 4,518,584 and 4,737,462.

As outlined above, in certain aspects, amino acid changes are made at specific sites in a lipocalin domain such as in the amino acid loops at the end of a beta-barrel structure or at positions exposed to the interior of the beta-barrel. In making such changes, the hydropathic index of amino acids may be considered. The importance of the hydropathic amino acid index in conferring interactive biological function on a protein is generally understood in the art (e.g., Kyte and Doolittle, 1982). It is accepted that the relative hydropathic character of the amino acid contributes to the secondary structure of the resultant protein, which in turn defines the interaction of the protein with other molecules, for example, enzymes, substrates, receptors, DNA, antibodies, antigens, and the like.

Each amino acid may be assigned a hydropathic index on the basis of their hydrophobicity and charge characteristics. These are, for instance: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate/glutamine/aspartate/asparagine (−3.5); lysine (−3.9); and arginine (−4.5). It is known in the art that certain amino acids may be substituted by other amino acids having a similar hydropathic index or score and still result in a protein with similar biological activity, i.e., still obtain a biologically functional protein. In making such changes, the substitution of amino acids whose hydropathic indices are within +/−2, those within +/−1 are or those within +/−0.5 would represent conservative substitutions.

It is also understood in the art that the substitution of like amino acids may be made effectively on the basis of hydrophilicity. U.S. Pat. No. 4,554,101 states that the greatest local average hydrophilicity of a protein, as governed by the hydrophilicity of its adjacent amino acids, correlates with a biological property of the protein. The following hydrophilicity values have been assigned to amino acids: arginine/lysine (+3.0); aspartate/glutamate (+3.0.+−0.1); serine (+0.3); asparagine/glutamine (+0.2); glycine (0); threonine (−0.4); proline (−0.5.+−0.1); alanine/histidine (−0.5); cysteine (−1.0); methionine (−1.3); valine (−1.5); leucine/isoleucine (−1.8); tyrosine (−2.3); phenylalanine (−2.5); and tryptophan (−3.4).

As outlined above, amino acid substitutions are therefore based on the relative similarity of the amino acid side-chain substituents, for example, their hydrophobicity, hydrophilicity, charge, size, and the like. Exemplary substitutions which take several of the foregoing characteristics into consideration are well known to those of skill in the art and include: arginine and lysine; glutamate and aspartate; serine and threonine; glutamine and asparagine; and valine, leucine, and isoleucine.

It is understood that the temperature and ionic strength of a desired stringency are determined in part by the length of the particular nucleic acid(s), the length and nucleobase content of the target sequence(s), the charge composition of the nucleic acid(s), and to the presence or concentration of formamide, tetramethylammonium chloride or other solvent(s) in a hybridization mixture. It is also understood that compositions and conditions for hybridization are mentioned by way of non-limiting examples only, and that the desired stringency for a particular hybridization reaction in a plant cell is often determined empirically by comparison to one or more positive or negative controls. Depending on the application envisioned varying conditions of hybridization may be employed to achieve varying degrees of selectivity of a nucleic acid towards a target sequence.

### II. MODIFICATION OF ALPHA-HELIX COILED-COIL FORMING POLYPEPTIDE CODING SEQUENCES

In certain aspects, the coding sequence for an alpha-helix coiled-coil forming polypeptide may be altered, for example to allow for enhanced expression or efficacy in a plant. For instance, codon frequencies may be adjusted, or regulatory sequences, such as a tissue specific promoter, enhancer, intron, or other regulatory sequence, may be fused to the sequence encoding the coiled coil alpha helices, such as the heptad repeat sequences within any of SEQ ID NOs: 20-25. A localization sequence such as a vacuolar or chloroplast transit peptide may also be added. Further, the sequences encoding a binding domain are added, as well as, for instance, sequences encoding a toxin domain, or other domain which functions to interfere with growth or replication of a plant pathogen or plant pest. The resulting protein can thus be made to accommodate binding of molecular targets of different size, shape, and chemical character. The binding of such molecules is contemplated to be to receptors on the surfaces of cells within the gut of an insect or other plant pest or pathogen that may infest crop plants, and should be compatible with the binding of other toxins that may be co-presented to the gut or cell surfaces, independently binding different receptors for the purpose of providing a means for resistance management, i.e., enhancing the resistance management practice embodied by the presence of two or more toxic molecules that are each different from each other and co-presented to the said surface. Alternatively, the alpha helical structures, such as alphabodies, could be engineered to bind to a particular molecule in a plant pest and as a consequence of the binding prevent the degradation or modification of a composition or agent or chemical that would otherwise act as a toxin when consumed by the pest, the binding effectively allowing the toxin composition to increase in effectiveness due to the inhibition by the engineered alphabody of the target molecule that exhibits toxin degradative or inhibitory properties.

### III. TARGET MOLECULES

As detailed above recombinant plant beta-barrel binding proteins or alpha-helical coiled-coil polypeptides can be produced that bind with high affinity and specificity to a wide array of a target molecules. The target molecule can be a protein, a carbohydrate, a nucleic acid (e.g., a double-stranded nucleic acid) or a small molecule. Target molecules may be specific to particular plant pests. For example, carbohydrates and proteins found on the surface of insect cells, fungi, bacteria and viruses may be used. In certain embodiments, the target molecule is a molecule expressed in an insect midgut. A target molecule to be bound by a particular anticalin or OB-domain protein can be any number of molecules including DNA or RNA segments normally targeted for binding by expression regulating proteins, for example, operator segments, enhancer segments, intron splice recognition segments and the like. Also, expression regulatory proteins such as gyrases, helicases, polymerases, ribosomal proteins and ribosome binding features can be emulated by such beta-barrel binding protein. Emulation brought about by the binding characteristics of such beta-barrel binding protein, particularly when expressed in a transgenic plant or plant cell, can result in modulated regulation of certain genes or pathways targeted for differential expression and thus bring about the increase or decrease in a particular desirable trait to the plant or plant tissue composed of such plant cells.

In some embodiments, beta-barrel binding proteins or alpha-helical coiled-coil polypeptides are produced for bringing about the increases or decrease in any particular desirable trait to the plant or plant tissue composed of such plant cells can be combined together with other transgenes that provide similar effects. For example, a plant derived beta-barrel binding protein or alpha-helical coiled-coil polypeptide that binds a particular receptor protein or glycoprotein in the gut of an a target insect pest of a plant can be coupled to a segment of an insecticidal protein that does not itself convey insecticidal properties, but which, when linked to such beta-barrel binding protein or alpha-helical coiled-coil polypeptide and allowed to bind to such receptor, brings about an insecticidal effect upon such insect pest. Such proteins can be used in combination with other different insecticidal proteins or insecticidal chemical agents to overcome the development of resistance to any single particular toxin protein or chemical agent.

In some embodiments, the target molecule is a polypeptide such as a peritrophin, chitinase, aminopeptidase, cystein protease, trypsin, chymotrypsin or carboxypeptidase.

Small molecule targets include, for example, ACCase inhibitors, ALS inhibitors, triazines, bypyridiliums, synthetic auxins, glycines, triketones, or herbicides. Table 2 provides a list of example small molecule targets for plant beta-barrel binding proteins.

TABLE 2
Small molecule beta-barrel binding protein targets

| Common Name | Trade Name | Formulation | Manufacturer |
|---|---|---|---|
| 2,4-D | FORMULA 40 | 3.67E | Nufarm |
| 2,4-DB | 2,4-DB | various | several |
| acetochlor | SURPASS | 6.4E | Dow AgroSciences |
| acifluorfen | BLAZER | 2E | United Phosphorus |
| alachlor | MICRO-TECH | 4E | Monsanto |
| ametryn | EVIK | 76WDG | Syngenta |
| atrazine | AATREX | 4L | Syngenta |
| aminopyralid | MILESTONE | 2E | Dow AgroSciences |
| benefin | BALAN | 60DF | UAP - Loveland |
| bensulfuron | LONDAX | 60WG | DuPont |
| bensulide | PREFAR | 4E | Gowan |
| bentazon | BASAGRAN | 4L | several |
| bromacil | HYVAR-X | 80WP | DuPont |
| bromoxynil | BUCTRIL | 2E | Bayer CropScience |
| butylate | SUTAN+ | 6.7E | Helm AgroScience |
| carfentrazone | AIM | 40DF | FMC |
| chlorimuron | CLASSIC | 25WG | DuPont |
| chlorsulfuron | GLEAN | 75DF | DuPont |
| clethodim | SELECT | 2E | several |
| clomazone | COMMAND | 3ME | several |
| clopyralid | STINGER | 3E | Dow AgroSciences |
| cloransulam | FIRSTRATE | 84WDG | Dow AgroSciences |
| cycloate | RO-NEET | 6E | Helm AgroScience |
| DCPA | DACTHAL | 75W | Amvac |
| desmedipham | BETANEX | 1.3L | Bayer CropScience |
| dicamba | CLARITY | 4L | BASF |
| dichlobenil | CASORON | 4G | Chemtura |
| diclofop | HOELON | 3E | Bayer CropScience |
| diclosulam | STRONGARM | 0.84E | Dow AgroScienes |
| diflufenzopyr | DISTINCT | 70WG | BASF |
| dimethenamid | OUTLOOK | 6E | BASF |
| diquat | REGLONE | 2E | Syngenta |
| diuron | KARMEX | 80DF | Griffin |
| DSMA | DSMA | 4L | several |
| endothall | DESICATE | 0.52E | Cerexagri Inc |
| EPTC | EPTAM | 7E | Gowan |
| ethalfluralin | CURBIT | 3E | UAP - Loveland |
| ethofumesate | NORTRON | 4SC | Bayer CropScience |
| fenoxaprop | PUMA | 1E | Bayer CropScience |

### IV. PLANT TRANSFORMATION CONSTRUCTS

Certain embodiments of the current invention concern plant transformation constructs. For example, one aspect of the current invention is a plant transformation vector comprising a recombinant plant beta-barrel binding protein coding sequence or alpha-helical coiled-coil polypeptide coding sequence alone, or in combination with one or more additional genes of agronomic interest. Such coding sequences may be present in one or more plant expression cassettes and/or transformation vectors for introduction to a plant cell. In certain embodiments of the invention, coding sequences are provided operably linked to a promoter (e.g., a heterologous). Expression constructs are also provided comprising these sequences, as are plants and plant cells transformed with the sequences.

The construction of vectors which may be employed in conjunction with plant transformation techniques using these or other sequences according to the invention will be known to those of skill of the art in light of the present disclosure (see, for example, Sambrook et al., 1989; Gelvin et al., 1990). The techniques of the current invention are thus not limited to any particular nucleic acid sequences.

One important use of the sequences provided by the invention will be in the alteration of plant phenotypes by genetic transformation. The recombinant plant beta-barrel binding protein coding sequences or alpha-helical coiled-coil polypeptide coding sequences may be provided with other sequences. Where an expressible coding region that is not necessarily a marker coding region is employed in combination with a marker coding region, one may employ the separate coding regions on either the same or different DNA segments for transformation. In the latter case, the different vectors are delivered concurrently to recipient cells to maximize cotransformation.

The choice of any additional elements used in conjunction with the recombinant plant beta-barrel binding protein coding sequences or alpha-helical coiled-coil polypeptide coding sequences will often depend on the purpose of the transformation. One of the major purposes of transformation of crop plants is to add commercially desirable, agronomically important traits to the plant. DNA segments used for transforming plant cells will, of course, generally comprise the cDNA, gene or genes which one desires to introduce into and have expressed in the host cells. These DNA segments can further include structures such as promoters, enhancers, polylinkers, or even regulatory genes as desired. The DNA segment or gene chosen for cellular introduction will often encode a protein which will be expressed in the resultant recombinant cells resulting in a screenable or selectable trait and/or which will impart an improved phenotype to the resulting transgenic plant. However, this may not always be the case, and the present invention also encompasses transgenic plants incorporating non-expressed transgenes. Components likely to be included with vectors used in the current invention are as follows.

A. Regulatory Elements

Exemplary promoters for expression of a nucleic acid sequence include plant promoter such as the CaMV 35S promoter (Odell et al., 1985), or others such as CaMV 19S (Lawton et al., 1987), nos (Ebert et al., 1987), Adh (Walker et al., 1987), sucrose synthase (Yang and Russell, 1990), α-tubulin, actin (Wang et al., 1992), cab (Sullivan et al., 1989), PEPCase

(Hudspeth and Grula, 1989) or those associated with the R gene complex (Chandler et al., 1989). Tissue specific promoters such as root cell promoters (Conkling et al., 1990) and tissue specific enhancers (Fromm et al., 1986) are also contemplated to be particularly useful, as are inducible promoters such as ABA- and turgor-inducible promoters.

The DNA sequence between the transcription initiation site and the start of the coding sequence, i.e., the untranslated leader sequence, can also influence gene expression. One may thus wish to employ a particular leader sequence with a transformation construct of the invention. Leader sequences are contemplated to include those which comprise sequences predicted to direct optimum expression of the attached gene, i.e., to include a consensus leader sequence which may increase or maintain mRNA stability and prevent inappropriate initiation of translation. The choice of such sequences will be known to those of skill in the art in light of the present disclosure. Sequences that are derived from genes that are highly expressed in plants may provide the most robust activity.

It is specifically envisioned that recombinant plant beta-barrel binding protein coding sequences or alpha-helical coiled-coil polypeptide coding sequences may be introduced under the control of novel promoters or enhancers, etc., or homologous or tissue specific promoters or control elements. Vectors for use in tissue-specific targeting of genes in transgenic plants will typically include tissue-specific promoters and may also include other tissue-specific control elements such as enhancer sequences. Promoters which direct specific or enhanced expression in certain plant tissues will be known to those of skill in the art in light of the present disclosure. These include, for example, the rbcS promoter, specific for green tissue; the ocs, nos and mas promoters which have higher activity in roots or wounded leaf tissue; a truncated (−90 to +8) 35S promoter which directs enhanced expression in roots, and an α-tubulin gene that also directs expression in roots.

B. Terminators

Transformation constructs prepared in accordance with the invention will typically include a 3′ end DNA sequence that acts as a signal to terminate transcription and allow for the poly-adenylation of the mRNA produced by coding sequences operably linked to a recombinant plant beta-barrel binding protein coding sequence or alpha-helical coiled-coil polypeptide coding sequence. A heterologous 3′ end (relative to the alpha-helical coiled-coil polypeptide or lipocalin or OB-domain protein from which the beta-barrel binding protein was derived) may enhance the expression of recombinant plant alpha-helical coiled-coil polypeptide or beta-barrel binding protein coding sequences. Terminators which are deemed to be particularly useful in this context include those from the nopaline synthase gene of Agrobacterium tumefaciens (nos 3′ end) (Bevan et al., 1983), the terminator for the T7 transcript from the octopine synthase gene of Agrobacterium tumefaciens, and the 3′ end of the protease inhibitor I or II genes from potato or tomato. Regulatory elements such as an Adh intron (Callis et al., 1987), sucrose synthase intron (Vasil et al., 1989) or TMV omega element (Gallie et al., 1989), may further be included where desired.

C. Transit or Signal Peptides

Sequences that are joined to the coding sequence of an expressed gene, which are removed post-translationally from the initial translation product and which facilitate the transport of the protein into or through intracellular or extracellular membranes, are termed transit (usually into vacuoles, vesicles, plastids and other intracellular organelles) and signal sequences (usually to the endoplasmic reticulum, golgi apparatus and outside of the cellular membrane). By facilitating the transport of the protein into compartments inside and outside the cell, these sequences may increase the accumulation of gene product protecting them from proteolytic degradation. These sequences also allow for additional mRNA sequences from highly expressed genes to be attached to the coding sequence of the genes. Since mRNA being translated by ribosomes is more stable than naked mRNA, the presence of translatable mRNA in front of the gene may increase the overall stability of the mRNA transcript from the gene and thereby increase synthesis of the gene product. Since transit and signal sequences are usually post-translationally removed from the initial translation product, the use of these sequences allows for the addition of extra translated sequences that may not appear on the final polypeptide. It further is contemplated that targeting of certain proteins may be desirable in order to enhance the stability of the protein (U.S. Pat. No. 5,545,818, incorporated herein by reference in its entirety).

Additionally, vectors may be constructed and employed in the intracellular targeting of a specific gene product within the cells of a transgenic plant or in directing a protein to the extracellular environment. This generally will be achieved by joining a DNA sequence encoding a transit or signal peptide sequence to the coding sequence of a particular gene. The resultant transit, or signal, peptide will transport the protein to a particular intracellular, or extracellular destination, respectively, and will then be post-translationally removed.

D. Marker Genes

By employing a selectable or screenable marker protein, one can provide or enhance the ability to identify transformants. “Marker genes” are genes that impart a distinct phenotype to cells expressing the marker protein and thus allow such transformed cells to be distinguished from cells that do not have the marker. Such genes may encode either a selectable or screenable marker, depending on whether the marker confers a trait which one can “select” for by chemical means, i.e., through the use of a selective agent (e.g., a herbicide, antibiotic, or the like), or whether it is simply a trait that one can identify through observation or testing, i.e., by “screening” (e.g., the green fluorescent protein). Of course, many examples of suitable marker proteins are known to the art and can be employed in the practice of the invention.

Included within the terms “selectable” or “screenable markers” also are genes which encode a “secretable marker” whose secretion can be detected as a means of identifying or selecting for transformed cells. Examples include markers which are secretable antigens that can be identified by antibody interaction, or even secretable enzymes which can be detected by their catalytic activity. Secretable proteins fall into a number of classes, including small, diffusible proteins detectable, e.g., by ELISA; small active enzymes detectable in extracellular solution (e.g., α-amylase, β-lactamase, phosphinothricin acetyltransferase); and proteins that are inserted or trapped in the cell wall (e.g., proteins that include a leader sequence such as that found in the expression unit of extensin or tobacco PR-S).

With regard to selectable secretable markers, the use of a gene that encodes a protein that becomes sequestered in the cell wall, and which protein includes a unique epitope is considered to be particularly advantageous. Such a secreted antigen marker would ideally employ an epitope sequence that would provide low background in plant tissue, a promoter-leader sequence that would impart efficient expression and targeting across the plasma membrane, and would produce protein that is bound in the cell wall and yet accessible to antibodies. A normally secreted wall protein modified to include a unique epitope would satisfy all such requirements.

Many selectable marker coding regions are known and could be used with the present invention including, but not limited to, neo (Potrykus et al., 1985), which provides kanamycin resistance and can be selected for using kanamycin, G418, paromomycin, etc.; bar, which confers bialaphos or phosphinothricin resistance; a mutant EPSP synthase protein (Hinchee et al., 1988) conferring glyphosate resistance; a nitrilase such as bxn from Klebsiella ozaenae which confers resistance to bromoxynil (Stalker et al., 1988); a mutant acetolactate synthase (ALS) which confers resistance to imidazolinone, sulfonylurea or other ALS inhibiting chemicals (European Patent Application 154,204, 1985); a methotrexate resistant DHFR (Thillet et al., 1988), a dalapon dehalogenase that confers resistance to the herbicide dalapon; or a mutated anthranilate synthase that confers resistance to 5-methyl tryptophan.

An illustrative embodiment of selectable marker capable of being used in systems to select transformants are those that encode the enzyme phosphinothricin acetyltransferase, such as the bar gene from Streptomyces hygroscopicus or the pat gene from Streptomyces viridochromogenes. The enzyme phosphinothricin acetyl transferase (PAT) inactivates the active ingredient in the herbicide bialaphos, phosphinothricin (PPT). PPT inhibits glutamine synthetase, (Murakami et al., 1986; Twell et al., 1989) causing rapid accumulation of ammonia and cell death.

Screenable markers that may be employed include a β-glucuronidase (GUS) or uidA gene which encodes an enzyme for which various chromogenic substrates are known; an R-locus gene, which encodes a product that regulates the production of anthocyanin pigments (red color) in plant tissues (Dellaporta et al., 1988); a β-lactamase gene (Sutcliffe, 1978), which encodes an enzyme for which various chromogenic substrates are known (e.g., PADAC, a chromogenic cephalosporin); a xylE gene (Zukowsky et al., 1983) which encodes a catechol dioxygenase that can convert chromogenic catechols; an α-amylase gene (Ikuta et al., 1990); a tyrosinase gene (Katz et al., 1983) which encodes an enzyme capable of oxidizing tyrosine to DOPA and dopaquinone which in turn condenses to form the easily-detectable compound melanin; a β-galactosidase gene, which encodes an enzyme for which there are chromogenic substrates; a luciferase (lux) gene (Ow et al., 1986), which allows for bioluminescence detection; an aequorin gene (Prasher et al., 1985) which may be employed in calcium-sensitive bioluminescence detection; or a gene encoding for green fluorescent protein (Sheen et al., 1995; Haseloff et al., 1997; Reichel et al., 1996; Tian et al., 1997; WO 97/41228).

Another screenable marker contemplated for use in the present invention is firefly luciferase, encoded by the lux gene. The presence of the lux gene in transformed cells may be detected using, for example, X-ray film, scintillation counting, fluorescent spectrophotometry, low-light video cameras, photon counting cameras or multiwell luminometry. It also is envisioned that this system may be developed for populational screening for bioluminescence, such as on tissue culture plates, or even for whole plant screening. The gene which encodes green fluorescent protein (GFP) is also contemplated as a particularly useful reporter gene (Sheen et al., 1995; Haseloff et al., 1997; Reichel et al., 1996; Tian et al., 1997; WO 97/41228). Expression of green fluorescent protein may be visualized in a cell or plant as fluorescence following illumination by particular wavelengths of light.

### V. METHODS FOR GENETIC TRANSFORMATION

Suitable methods for transformation of plant or other cells for use with the current invention are believed to include virtually any method by which DNA can be introduced into a cell, such as by direct delivery of DNA such as by PEG-mediated transformation of protoplasts (Omirulleh et al., 1993), by desiccation/inhibition-mediated DNA uptake (Potrykus et al., 1985), by electroporation (U.S. Pat. No. 5,384,253, specifically incorporated herein by reference in its entirety), by agitation with silicon carbide fibers (Kaeppler et al., 1990; U.S. Pat. No. 5,302,523, specifically incorporated herein by reference in its entirety; and U.S. Pat. No. 5,464,765, specifically incorporated herein by reference in its entirety), by Agrobacterium-mediated transformation (U.S. Pat. No. 5,591,616 and U.S. Pat. No. 5,563,055; both specifically incorporated herein by reference) and by acceleration of DNA coated particles (U.S. Pat. No. 5,550,318; U.S. Pat. No. 5,538,877; and U.S. Pat. No. 5,538,880; each specifically incorporated herein by reference in its entirety), etc. Through the application of techniques such as these, the cells of virtually any plant species may be stably transformed, and these cells developed into transgenic plants.

A. Agrobacterium-Mediated Transformation

Agrobacterium-mediated transfer is a widely applicable system for introducing genes into plant cells because the DNA can be introduced into whole plant tissues, thereby bypassing the need for regeneration of an intact plant from a protoplast. The use of Agrobacterium-mediated plant integrating vectors to introduce DNA into plant cells is well known in the art. See, for example, the methods described by Fraley et al., (1985), Rogers et al., (1987) and U.S. Pat. No. 5,563,055, specifically incorporated herein by reference in its entirety.

Agrobacterium-mediated transformation is most efficient in dicotyledonous plants and is an efficient method for transformation of dicots, including Arabidopsis, tobacco, tomato, alfalfa and potato. Indeed, while Agrobacterium-mediated transformation has been routinely used with dicotyledonous plants for a number of years, it has only recently become applicable to monocotyledonous plants. Advances in Agrobacterium-mediated transformation techniques have now made the technique applicable to nearly all monocotyledonous plants. For example, Agrobacterium-mediated transformation techniques have now been applied to rice (Hiei et al., 1997; U.S. Pat. No. 5,591,616), wheat (McCormac et al., 1998), barley (Tingay et al., 1997; McCormac et al., 1998), alfalfa (e.g., Thomas et al., 1990; McKersie et al., 1993) and maize (Ishida et al., 1996).

Modern Agrobacterium transformation vectors are capable of replication in E. coli as well as Agrobacterium, allowing for convenient manipulations as described (Klee et al., 1985). Moreover, recent technological advances in vectors for Agrobacterium-mediated gene transfer have improved the arrangement of genes and restriction sites in the vectors to facilitate the construction of vectors capable of expressing various polypeptide coding genes. The vectors described (Rogers et al., 1987) have convenient multi-linker regions flanked by a promoter and a polyadenylation site for direct expression of inserted polypeptide coding genes and are suitable for present purposes. In addition, Agrobacterium containing both armed and disarmed Ti genes can be used for the transformations. In those plant strains where Agrobacterium-mediated transformation is efficient, it is the method of choice because of the facile and defined nature of the gene transfer.

B. Electroporation

To effect transformation by electroporation, one may employ either friable tissues, such as a suspension culture of cells or embryogenic callus or alternatively one may transform immature embryos or other organized tissue directly. In this technique, one would partially degrade the cell walls of the chosen cells by exposing them to pectin-degrading enzymes (pectolyases) or mechanically wounding in a controlled manner. Examples of some species which have been transformed by electroporation of intact cells include maize (U.S. Pat. No. 5,384,253; Rhodes et al., 1995; D'Halluin et al., 1992), wheat (Zhou et al., 1993), tomato (Hou and Lin, 1996), soybean (Christou et al., 1987) and tobacco (Lee et al., 1989).

One also may employ protoplasts for electroporation transformation of plants (Bates, 1994; Lazzeri, 1995). For example, the generation of transgenic soybean plants by electroporation of cotyledon-derived protoplasts is described by Dhir and Widholm in Intl. Patent Appl. Publ. No. WO 9217598 (specifically incorporated herein by reference). Other examples of species for which protoplast transformation has been described include barley (Lazerri, 1995), sorghum (Battraw et al., 1991), maize (Bhattacharjee et al., 1997), wheat (He et al., 1994) and tomato (Tsukada, 1989).

C. Microprojectile Bombardment

Another method for delivering transforming DNA segments to plant cells in accordance with the invention is microprojectile bombardment (U.S. Pat. No. 5,550,318; U.S. Pat. No. 5,538,880; U.S. Pat. No. 5,610,042; and PCT Application WO 94/09699; each of which is specifically incorporated herein by reference in its entirety). In this method, particles may be coated with nucleic acids and delivered into cells by a propelling force. Exemplary particles include those comprised of tungsten, platinum, and gold. It is contemplated that in some instances DNA precipitation onto metal particles would not be necessary for DNA delivery to a recipient cell using microprojectile bombardment. However, it is contemplated that particles may contain DNA rather than be coated with DNA. Hence, it is proposed that DNA-coated particles may increase the level of DNA delivery via particle bombardment but are not, in and of themselves, necessary.

For the bombardment, cells in suspension are concentrated on filters or solid culture medium. Alternatively, immature embryos or other target cells may be arranged on solid culture medium. The cells to be bombarded are positioned at an appropriate distance below the macroprojectile stopping plate.

An illustrative embodiment of a method for delivering DNA into plant cells by acceleration is the Biolistics® Particle Delivery System (Dupont), which can be used to propel particles coated with DNA or cells through a screen, such as a stainless steel or nylon screen (e.g., NYTEX screen; Sefar America, Depew, NY USA), onto a filter surface covered with plant cells cultured in suspension. The screen disperses the particles so that they are not delivered to the recipient cells in large aggregates. Microprojectile bombardment techniques are widely applicable, and may be used to transform virtually any plant species. Examples of species for which have been transformed by microprojectile bombardment include monocot species such as maize (PCT Application WO 95/06128), barley (Ritala et al., 1994), wheat (U.S. Pat. No. 5,563,055), and sorghum (Casa et al., 1993); as well as a number of dicots including tobacco (Tomes et al., 1990; Buising and Benbow, 1994), soybean (U.S. Pat. No. 5,322,783), sunflower (Knittel et al., 1994), peanut (Singsit et al., 1997), cotton (McCabe and Martinell, 1993), tomato (VanEck et al., 1995), and legumes in general (U.S. Pat. No. 5,563,055, specifically incorporated herein by reference in its entirety).

D. Other Transformation Methods

Transformation of protoplasts can be achieved using methods based on calcium phosphate precipitation, polyethylene glycol treatment, electroporation, and combinations of these treatments (see, e.g., Potrykus et al., 1985; Lorz et al., 1985; Omirulleh et al., 1993; Fromm et al., 1986; Uchimiya et al., 1986; Callis et al., 1987; Marcotte et al., 1988).

Application of these systems to different plant strains depends upon the ability to regenerate that particular plant strain from protoplasts. Illustrative methods for the regeneration of plants from protoplasts have been described (Toriyama et al., 1986; Yamada et al., 1986; Abdullah et al., 1986; Omirulleh et al., 1993 and U.S. Pat. No. 5,508,184). Examples of the use of direct uptake transformation of protoplasts include transformation of rice (Ghosh-Biswas et al., 1994), sorghum (Battraw and Hall, 1991), barley (Lazerri, 1995), oat (Zheng and Edwards, 1990) and maize (Omirulleh et al., 1993).

To transform plant strains that cannot be successfully regenerated from protoplasts, other ways to introduce DNA into intact cells or tissues can be utilized. For example, regeneration of cereals from immature embryos or explants can be effected as described (Vasil, 1989). Also, silicon carbide fiber-mediated transformation may be used with or without protoplasting (Kaeppler, 1990; Kaeppler et al., 1992; U.S. Pat. No. 5,563,055). Transformation with this technique is accomplished by agitating silicon carbide fibers together with cells in a DNA solution. DNA passively enters as the cells are punctured. This technique has been used successfully with, for example, the monocot cereals maize (PCT Application WO 95/06128; (Thompson, 1995) and rice (Nagatani, 1997).

### VI. PRODUCTION AND CHARACTERIZATION OF STABLY TRANSFORMED PLANTS

After effecting delivery of exogenous DNA to recipient cells, the next steps generally concern identifying the transformed cells for further culturing and plant regeneration. In order to improve the ability to identify transformants, one may desire to employ a selectable or screenable marker gene with a transformation vector prepared in accordance with the invention. In this case, one would then generally assay the potentially transformed cell population by exposing the cells to a selective agent or agents, or one would screen the cells for the desired marker gene trait.

A. Selection

It is believed that DNA is introduced into only a small percentage of target cells in any one experiment. In order to provide an efficient system for identification of those cells receiving DNA and integrating it into their genomes one may employ a means for selecting those cells that are stably transformed. One exemplary embodiment of such a method is to introduce into the host cell, a marker gene which confers resistance to some normally inhibitory agent, such as an antibiotic or herbicide. Examples of antibiotics which may be used include the aminoglycoside antibiotics neomycin, kanamycin and paromomycin, or the antibiotic hygromycin. Resistance to the aminoglycoside antibiotics is conferred by aminoglycoside phosphotransferase enzymes such as neomycin phosphotransferase II (NPT II) or NPT I, whereas resistance to hygromycin is conferred by hygromycin phosphotransferase.

Potentially transformed cells then are exposed to the selective agent. In the population of surviving cells will be those cells where, generally, the resistance-conferring gene has been integrated and expressed at sufficient levels to permit cell survival. Cells may be tested further to confirm stable integration of the exogenous DNA.

One herbicide which constitutes a desirable selection agent is the broad spectrum herbicide bialaphos. Bialaphos is a tripeptide antibiotic produced by Streptomyces hygroscopicus and is composed of phosphinothricin (PPT), an analogue of L-glutamic acid, and two L-alanine residues. Upon removal of the L-alanine residues by intracellular peptidases, the PPT is released and is a potent inhibitor of glutamine synthetase (GS), a pivotal enzyme involved in ammonia assimilation and nitrogen metabolism (Ogawa et al., 1973). Synthetic PPT, the active ingredient in the herbicide Liberty™ also is effective as a selection agent. Inhibition of GS in plants by PPT causes the rapid accumulation of ammonia and death of the plant cells.

The organism producing bialaphos and other species of the genus Streptomyces also synthesizes an enzyme phosphinothricin acetyl transferase (PAT) which is encoded by the bar gene in Streptomyces hygroscopicus and the pat gene in Streptomyces viridochromogenes. The use of the herbicide resistance gene encoding phosphinothricin acetyl transferase (PAT) is referred to in DE 3642 829 A, wherein the gene is isolated from Streptomyces viridochromogenes. In the bacterial source organism, this enzyme acetylates the free amino group of PPT preventing auto-toxicity (Thompson et al., 1987). The bar gene has been cloned (Murakami et al., 1986; Thompson et al., 1987) and expressed in transgenic tobacco, tomato, potato (De Block et al., 1987) Brassica (De Block et al., 1989) and maize (U.S. Pat. No. 5,550,318). In previous reports, some transgenic plants which expressed the resistance gene were completely resistant to commercial formulations of PPT and bialaphos in greenhouses.

Another example of a herbicide which is useful for selection of transformed cell lines in the practice of the invention is the broad spectrum herbicide glyphosate. Glyphosate inhibits the action of the enzyme EPSPS which is active in the aromatic amino acid biosynthetic pathway. Inhibition of this enzyme leads to starvation for the amino acids phenylalanine, tyrosine, and tryptophan and secondary metabolites derived thereof. U.S. Pat. No. 4,535,060 describes the isolation of EPSPS mutations which confer glyphosate resistance on the Salmonella typhimurium gene for EPSPS, aroA. The EPSPS gene was cloned from Zea mays and mutations similar to those found in a glyphosate resistant aroA gene were introduced in vitro. Mutant genes encoding glyphosate resistant EPSPS enzymes are described in, for example, International Patent WO 97/4103. The best characterized mutant EPSPS gene conferring glyphosate resistance comprises amino acid changes at residues 102 and 106, although it is anticipated that other mutations will also be useful (PCT/WO97/4103).

To use the bar-bialaphos or the EPSPS-glyphosate selective system, transformed tissue is cultured for 0-28 days on nonselective medium and subsequently transferred to medium containing from 1-3 mg/1 bialaphos or 1-3 mM glyphosate as appropriate. While ranges of 1-3 mg/1 bialaphos or 1-3 mM glyphosate will typically be employed, it is proposed that ranges of 0.1-50 mg/1 bialaphos or 0.1-50 mM glyphosate will find utility.

It further is contemplated that the herbicide DALAPON, 2,2-dichloropropionic acid, may be useful for identification of transformed cells. The enzyme 2,2-dichloropropionic acid dehalogenase (deh) inactivates the herbicidal activity of 2,2-dichloropropionic acid and therefore confers herbicidal resistance on cells or plants expressing a gene encoding the dehalogenase enzyme (Buchanan-Wollaston et al., 1992; U.S. Pat. No. 5,508,468).

Alternatively, a gene encoding anthranilate synthase, which confers resistance to certain amino acid analogs, e.g., 5-methyltryptophan or 6-methyl anthranilate, may be useful as a selectable marker gene. The use of an anthranilate synthase gene as a selectable marker was described in U.S. Pat. No. 5,508,468.

An example of a screenable marker trait is the enzyme luciferase. In the presence of the substrate luciferin, cells expressing luciferase emit light which can be detected on photographic or x-ray film, in a luminometer (or liquid scintillation counter), by devices that enhance night vision, or by a highly light sensitive video camera, such as a photon counting camera. These assays are nondestructive and transformed cells may be cultured further following identification. The photon counting camera is especially valuable as it allows one to identify specific cells or groups of cells which are expressing luciferase and manipulate those in real time. Another screenable marker which may be used in a similar fashion is the gene coding for green fluorescent protein.

It further is contemplated that combinations of screenable and selectable markers will be useful for identification of transformed cells. In some cell or tissue types a selection agent, such as bialaphos or glyphosate, may either not provide enough killing activity to clearly recognize transformed cells or may cause substantial nonselective inhibition of transformants and nontransformants alike, thus causing the selection technique to not be effective. It is proposed that selection with a growth inhibiting compound, such as bialaphos or glyphosate at concentrations below those that cause 100% inhibition followed by screening of growing tissue for expression of a screenable marker gene such as luciferase would allow one to recover transformants from cell or tissue types that are not amenable to selection alone. It is proposed that combinations of selection and screening may enable one to identify transformants in a wider variety of cell and tissue types. This may be efficiently achieved using a gene fusion between a selectable marker gene and a screenable marker gene, for example, between an NPTII gene and a GFP gene.

B. Regeneration and Seed Production

Cells that survive the exposure to the selective agent, or cells that have been scored positive in a screening assay, may be cultured in media that supports regeneration of plants. In an exemplary embodiment, MS and N6 media may be modified by including further substances such as growth regulators. One such growth regulator is dicamba or 2,4-D. However, other growth regulators may be employed, including NAA, NAA+2,4-D or picloram. Media improvement in these and like ways has been found to facilitate the growth of cells at specific developmental stages. Tissue may be maintained on a basic media with growth regulators until sufficient tissue is available to begin plant regeneration efforts, or following repeated rounds of manual selection, until the morphology of the tissue is suitable for regeneration, at least 2 wk, then transferred to media conducive to maturation of embryoids. Cultures are transferred every 2 wk on this medium. Shoot development will signal the time to transfer to medium lacking growth regulators.

The transformed cells, identified by selection or screening and cultured in an appropriate medium that supports regeneration, will then be allowed to mature into plants. Developing plantlets are transferred to soiless plant growth mix, and hardened, e.g., in an environmentally controlled chamber, for example, at about 85% relative humidity, 600 ppm CO₂, and 25-250 microeinsteins m⁻² s⁻¹ of light. Plants are matured either in a growth chamber or greenhouse. Plants can be regenerated from about 6 wk to 10 months after a transformant is identified, depending on the initial tissue. During regeneration, cells are grown on solid media in tissue culture vessels. Illustrative embodiments of such vessels are petri dishes and Plantcon™ containers (MP-ICN Biomedicals, Solon, OH, USA). Regenerating plants may be grown at about 19 to 28° C. After the regenerating plants have reached the stage of shoot and root development, they may be transferred to a greenhouse for further growth and testing.

Seeds on transformed plants may occasionally require embryo rescue due to cessation of seed development and premature senescence of plants. To rescue developing embryos, they are excised from surface-disinfected seeds 10-20 days post-pollination and cultured. An embodiment of media used for culture at this stage comprises MS salts, 2% sucrose, and 5.5 g/1 agarose. In embryo rescue, large embryos (defined as greater than 3 mm in length) are germinated directly on an appropriate media. Embryos smaller than that may be cultured for 1 wk on media containing the above ingredients along with 10⁻⁵ M abscisic acid and then transferred to growth regulator-free medium for germination.

C. Characterization

To confirm the presence of the exogenous DNA or “transgene(s)” in the regenerating plants, a variety of assays may be performed. Such assays include, for example, “molecular biological” assays, such as Southern and northern blotting and PCR; “biochemical” assays, such as detecting the presence of a protein product, e.g., by immunological means (ELISAs and Western blots) or by enzymatic function; plant part assays, such as leaf or root assays; and also, by analyzing the phenotype of the whole regenerated plant.

D. DNA Integration, RNA Expression and Inheritance

Genomic DNA may be isolated from cell lines or any plant parts to determine the presence of the exogenous gene through the use of techniques well known to those skilled in the art. Note, that intact sequences will not always be present, presumably due to rearrangement or deletion of sequences in the cell. The presence of DNA elements introduced through the methods of this invention may be determined, for example, by polymerase chain reaction (PCR). Using this technique, discreet fragments of DNA are amplified and detected by gel electrophoresis. This type of analysis permits one to determine whether a gene is present in a stable transformant, but does not prove integration of the introduced gene into the host cell genome. It is typically the case, however, that DNA has been integrated into the genome of all transformants that demonstrate the presence of the gene through PCR analysis. In addition, it is not typically possible using thermal amplification techniques to determine whether transformants have exogenous genes introduced into different sites in the genome, i.e., whether transformants are of independent origin. It is contemplated that using PCR techniques it would be possible to clone fragments of the host genomic DNA adjacent to an introduced gene.

Positive proof of DNA integration into the host genome and the independent identities of transformants may be determined using the technique of Southern hybridization. Using this technique specific DNA sequences that were introduced into the host genome and flanking host DNA sequences can be identified. Hence the Southern hybridization pattern of a given transformant serves as an identifying characteristic of that transformant. In addition it is possible through Southern hybridization to demonstrate the presence of introduced genes in high molecular weight DNA, i.e., confirm that the introduced gene has been integrated into the host cell genome. The technique of Southern hybridization provides information that is obtained using PCR, e.g., the presence of a gene, but also demonstrates integration into the genome and characterizes each individual transformant.

Whereas DNA analysis techniques may be conducted using DNA isolated from any part of a plant, RNA will only be expressed in particular cells or tissue types and hence it will be necessary to prepare RNA for analysis from these tissues. PCR techniques also may be used for detection and quantitation of RNA produced from introduced genes. In this application of PCR it is first necessary to reverse transcribe RNA into DNA, using enzymes such as reverse transcriptase, and then through the use of conventional PCR techniques amplify the DNA. In most instances PCR techniques, while useful, will not demonstrate integrity of the RNA product.

Further information about the nature of the RNA product may be obtained by northern blotting. This technique will demonstrate the presence of an RNA species and give information about the integrity of that RNA. The presence or absence of an RNA species also can be determined using dot or slot blot northern hybridizations. These techniques are modifications of northern blotting and will only demonstrate the presence or absence of an RNA species.

E. Gene Expression

While Southern blotting and PCR may be used to detect the gene(s) in question, they do not provide information as to whether the corresponding protein is being expressed. Expression may be evaluated by determining expression via transcript-profiling techniques such as by use of a microarray, and by specifically identifying the protein products of the introduced genes or evaluating the phenotypic changes brought about by their expression.

Assays for the production and identification of specific proteins may make use of physical-chemical, structural, functional, or other properties of the proteins. Unique physical-chemical or structural properties allow the proteins to be separated and identified by electrophoretic procedures, such as native or denaturing gel electrophoresis or isoelectric focusing, or by chromatographic techniques such as ion exchange or gel exclusion chromatography. The unique structures of individual proteins offer opportunities for use of specific antibodies to detect their presence in formats such as an ELISA assay. Combinations of approaches may be employed with even greater specificity such as western blotting in which antibodies are used to locate individual gene products that have been separated by electrophoretic techniques. Additional techniques may be employed to absolutely confirm the identity of the product of interest such as evaluation by amino acid sequencing following purification. Although these are among the most commonly employed, other procedures may be additionally used.

Assay procedures also may be used to identify the expression of proteins by their functionality, especially the ability of enzymes to catalyze specific chemical reactions involving specific substrates and products. These reactions may be followed by providing and quantifying the loss of substrates or the generation of products of the reactions by physical or chemical procedures. Examples are as varied as the enzyme to be analyzed and may include assays for PAT enzymatic activity by following production of radiolabeled acetylated phosphinothricin from phosphinothricin and ¹⁴C-acetyl CoA or for anthranilate synthase activity by following loss of fluorescence of anthranilate, to name two.

Very frequently the expression of a gene product is determined by evaluating the phenotypic results of its expression. These assays also may take many forms including but not limited to analyzing changes in the chemical composition, morphology, or physiological properties of the plant. Chemical composition may be altered by expression of genes encoding enzymes or storage proteins which change amino acid composition and may be detected by amino acid analysis, or by enzymes which change starch quantity which may be analyzed by near infrared reflectance spectrometry. Morphological changes may include greater stature or thicker stalks. Most often changes in response of plants or plant parts to imposed treatments are evaluated under carefully controlled conditions termed bioassays.

### VII. BREEDING PLANTS OF THE INVENTION

In addition to direct transformation of a particular plant genotype with a construct prepared according to the current invention, transgenic plants may be made by crossing a plant having a selected DNA of the invention to a second plant lacking the construct. For example, a selected CT biosynthesis gene can be introduced into a particular plant variety by crossing, without the need for ever directly transforming a plant of that given variety. Therefore, the current invention not only encompasses a plant directly transformed or regenerated from cells which have been transformed in accordance with the current invention, but also the progeny of such plants. As used herein the term “progeny” denotes the offspring of any generation of a parent plant prepared in accordance with the instant invention, wherein the progeny comprises a selected DNA construct prepared in accordance with the invention. “Crossing” a plant to provide a plant line having one or more added transgenes relative to a starting plant line, as disclosed herein, is defined as the techniques that result in a transgene of the invention being introduced into a plant line by crossing a starting line with a donor plant line that comprises a transgene of the invention. To achieve this one could, for example, perform the following steps:

(a) plant seeds of the first (starting line) and second (donor plant line that comprises a transgene of the invention) parent plants;

(b) grow the seeds of the first and second parent plants into plants that bear flowers;

(c) pollinate a flower from the first parent plant with pollen from the second parent plant; and

(d) harvest seeds produced on the parent plant bearing the fertilized flower.

Backcrossing is herein defined as the process including the steps of:

(a) crossing a plant of a first genotype containing a desired gene, DNA sequence or element to a plant of a second genotype lacking the desired gene, DNA sequence or element;

(b) selecting one or more progeny plant containing the desired gene, DNA sequence or element;

(c) crossing the progeny plant to a plant of the second genotype; and

(d) repeating steps (b) and (c) for the purpose of transferring a desired DNA sequence from a plant of a first genotype to a plant of a second genotype.

Introgression of a DNA element into a plant genotype is defined as the result of the process of backcross conversion. A plant genotype into which a DNA sequence has been introgressed may be referred to as a backcross converted genotype, line, inbred, or hybrid.

Similarly a plant genotype lacking the desired DNA sequence may be referred to as an unconverted genotype, line, inbred, or hybrid.

### VIII. DEFINITIONS

Alpha-helix coiled-coil forming polypeptide: A proteinaceous scaffold comprising one or more peptides which can form a thermodynamically stable antiparallel or parallel coiled-coil structure comprising associated alpha helices connected by linker loops. Such peptides may further comprise domains with binding functions and localization functions. Such binding can be to a target molecule consisting of a glycoprotein, a protein, a peptide, a lipid or phospholipid, a polynucleotide having a specific sequence or three dimensional configuration such as a stem loop, a dumbbell, a multistem-loop structure, or a linear structure. A function of the binding can be to inhibit the further binding of a second molecule different from the bound alpha helix coiled-coil forming polypeptide, inhibit an enzymatic function of the bound target molecule, to enhance the function or biological activity of a particular target molecule, to give effect to a linked peptide such as a pore forming polypeptide segment, and the like.

Beta-Barrel Binding Protein: A polypeptide comprising a beta-barrel protein structure and having a binding affinity for a target ligand, such as a small molecule, a protein, a carbohydrate or a nucleic acid. Beta-barrel binding proteins include, but are not limited to, anticalin proteins (e.g., lipocalins) and OB-domain proteins (see, e.g., Murzin, 1993).

Expression: The combination of intracellular processes, including transcription and translation undergone by a coding DNA molecule such as a structural gene to produce a polypeptide.

Genetic Transformation: A process of introducing a DNA sequence or construct (e.g., a vector or expression cassette) into a cell or protoplast in which that exogenous DNA is incorporated into a chromosome or is capable of autonomous replication.

Heterologous: A sequence which is not normally present in a given host genome in the genetic context in which the sequence is currently found. In this respect, the sequence may be native to the host genome, but be rearranged with respect to other genetic sequences within the host sequence. For example, a regulatory sequence may be heterologous in that it is linked to a different coding sequence relative to the native regulatory sequence.

Obtaining: When used in conjunction with a transgenic plant cell or transgenic plant, obtaining means either transforming a non-transgenic plant cell or plant to create the transgenic plant cell or plant, or planting transgenic plant seed to produce the transgenic plant cell or plant. Such a transgenic plant seed may be from an R₀ transgenic plant or may be from a progeny of any generation thereof that inherits a given transgenic sequence from a starting transgenic parent plant.

Recombinant: A polypeptide or nucleic acid that has been altered from its naturally occurring state. For example, a recombinant lipocalin coding sequence may encode 1 or more amino acid deletions, insertions or substitutions relative to a native plant lipocalin coding sequence.

Promoter: A recognition site on a DNA sequence or group of DNA sequences that provides an expression control element for a structural gene and to which RNA polymerase specifically binds and initiates RNA synthesis (transcription) of that gene.

R₀ transgenic plant: A plant that has been genetically transformed or has been regenerated from a plant cell or cells that have been genetically transformed.

Regeneration: The process of growing a plant from a plant cell (e.g., plant protoplast, callus or explant).

Selected DNA: A DNA segment which one desires to introduce into a plant genome by genetic transformation.

Transformation construct: A chimeric DNA molecule which is designed for introduction into a host genome by genetic transformation. Transformation constructs will comprise all of the genetic elements necessary to direct the expression of one or more exogenous genes. In particular embodiments of the instant invention, it may be desirable to introduce a transformation construct into a host cell in the form of an expression cassette. Transformed cell: A cell the DNA complement of which has been altered by the introduction of an exogenous DNA molecule into that cell.

Transgene: A segment of DNA which has been incorporated into a host genome or is capable of autonomous replication in a host cell and is capable of causing the expression of one or more coding sequences. Exemplary transgenes will provide the host cell, or plants regenerated therefrom, with a novel phenotype relative to the corresponding non-transformed cell or plant. Transgenes may be directly introduced into a plant by genetic transformation, or may be inherited from a plant of any previous generation which was transformed with the DNA segment.

Transgenic plant: A plant or progeny plant of any subsequent generation derived therefrom, wherein the DNA of the plant or progeny thereof contains an introduced exogenous DNA segment not naturally present in a non-transgenic plant of the same strain. The transgenic plant may additionally contain sequences which are native to the plant being transformed, but wherein the “exogenous” gene has been altered in order to alter the level or pattern of expression of the gene, for example, by use of one or more heterologous regulatory or other elements.

Vector: A DNA molecule capable of replication in a host cell and/or to which another DNA segment can be operatively linked so as to bring about replication of the attached segment. A plasmid is an exemplary vector.

### IX. EXAMPLES

The following examples are included to demonstrate preferred embodiments of the invention. It should be appreciated by those of skill in the art that the techniques disclosed in the examples which follow represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### Example 1

### Production of a Recombinant Plant Lipocalin

The coding region for a plant lipocalin domain is identified by comparison to a domain such as those provided in SEQ ID NOs: 3, 6 and 9. Such a comparison may be by primary structure or may employ a program that predicts secondary structure. The plant lipocalin domain is then subject to mutagenesis to produce a plurality of recombinant plant lipocalin sequences. These sequences are then used to generate a library such as a phage display library. The library is screened for sequences that bind to a target molecule. In the case of phage display, the phages providing the displayed recombinant lipocalins are selected for binding to the target molecules thereby enabling the bound phage to be isolated and the recombinant anticalin coding sequences identified.

Anticalin sequences that are found to bind the target molecule may be further subjected to iterative rounds of random or site directed mutagenesis followed by screening for binding to the target molecule. A protocol for such an iterative screening or selection method is provided as FIG. 1. By completing an iterative selection a recombinant plant anticalin can be produced that has high affinity and specificity for a target molecule.

### Example 2

### Production of Target Molecule-Binding Recombinant Plant OB-Domain Proteins

The coding region for a plant OB-domain is identified by comparison to an OB-domain protein such as SEQ ID NO: 17 or 19. Such a comparison may be by primary structure or may employ a program that predicts secondary structure. The plant OB-domain protein is synthesized under conditions where amino acids are randomized at selected amino acid positions. These proteins are pooled into a library. The library is screened for proteins that bind to an immobilized target molecule. For example, the library may be screened using a proteins microarray to isolate recombinant OB-domain proteins that bind to a plurality of different target molecules (see, e.g., Cinier et al., 2009). The polypeptide sequences of proteins binding to targets of interest are determined by mass spectroscopy.

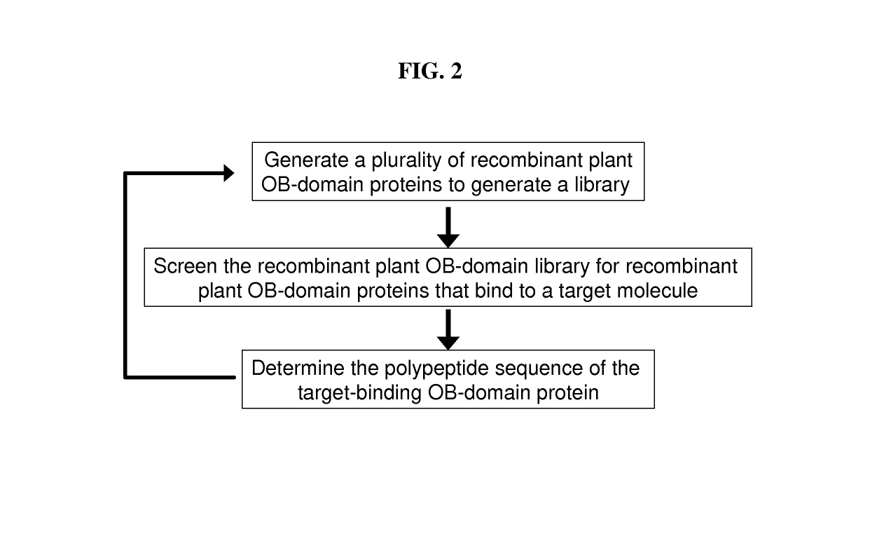

Recombinant OB-domain proteins that are found to bind to a target molecule may be further subjected to iterative rounds of selection by, again, synthesizing the recombinant protein under conditions that randomize amino acid incorporation at selected positions. A pool of such further modified recombinant proteins can be screened for enhanced binding to targets. An example protocol for such an iterative screening or selection method is provided as FIG. 2.

### Example 3

### Function of an Alpha-Helix-Forming Polypeptide in a Plant

Recombinant polypeptides have been found to form a central coil of associated alpha-helices connected by linker loops, in a parallel or an anti-parallel format (e.g. EP 2161278A1; WO2010066740). This three-dimensional “scaffold” structure provides the polypeptide with functional features including thermodynamic stability and flexibility for engineering the sequence encoding the proteinaceous scaffold to further allow for binding of one or more specific molecular target(s). For instance, the coding sequence for an alpha-helix coiled-coil forming polypeptide may be engineered in a linker region to comprise a sequence coding for a localization domain and/or a domain with a beneficial function in a plant. Such functions may include the ability to kill or prevent growth, replication, differentiation, or pathogenesis of an organism that can cause disease in, be a pest of, or otherwise affect (e.g. reduce) the yield of a crop plant. The alpha-helix coiled-coil forming polypeptide configuration may be monomeric (a single polypeptide chain with an alpha helix forming (“binding”) domain and one or more additional domains of interest). Alternatively, the alpha-helix coiled-coil forming polypeptide may function as a dimer (with two binding domains), or as a trimer (with three binding domains). Such alpha-helix coiled-coil forming polypeptides may be expressed in planta from a transgene, or may be applied topically to a plant part, such as a leaf, flower, seed, or stem following their expression in a cell culture system outside of a plant.

The alphabody or alpha-helix coiled-coil forming polypeptide can be fused to any other peptide molecule and used to localize the fused polypeptide to a particular region within or on the surface of a particular cell or cell type, whether in a plant cell or to the surface of a cell of a pest that consumes the fusion protein. The localization of the fusion or alpha-helix coiled-coil forming polypeptide can result in the death, stunting, or inhibition or alternatively, the enhancement of an organism that consumes the fusion or alpha-helix coiled-coil forming polypeptide in its diet, or the regulation or control of a target molecule to which the fusion or alpha-helix coiled-coil forming polypeptide binds. The fusion or alpha-helix coiled-coil forming polypeptide in a plant can result in the death, stunting, or inhibition or alternatively, the enhancement of an organism that consumes in its diet or otherwise is contacted by a plant comprising the fusion or alpha-helix coiled-coil forming polypeptide; and can result in the conferring upon the plant of a trait of agronomic interest selected from the group consisting of herbicide tolerance, drought resistance, insect resistance, fungus resistance, virus resistance, bacteria resistance, male sterility, cold tolerance, salt tolerance, increased yield, enhanced oil composition, increased oil content, enhanced nutrient use efficiency and altered amino acid content.

### REFERENCES

The references listed below are incorporated herein by reference to the extent that they supplement, explain, provide a background for, or teach methodology, techniques, and/or compositions employed herein.
U.S. Pat. Nos. 4,518,584; 4,535,060; 4,554,101; 4,737,462; 5,302,523; 5,322,783; 5,384,253; 5,464,765; 5,508,184; 5,508,468; 5,538,877; 5,538,880; 5,538,880; 5,545,818; 5,550,318; 5,563,055; 5,591,616; 5,610,042; 7,094,571; 7,250,297
U.S. Patent Application Publ. No. 20100305304
Abdullah et al., Biotechnology, 4:1087, 1986.
Arcus, Curr. Opin. Struct. Biol., 12:794-901, 2002.
Bates, Mol. Biotechnol., 2(2):135-145, 1994.
Battraw et al., Theor. App. Genet., 82(2):161-168, 1991.
Bevan et al., Nucleic Acids Research, 11(2):369-385, 1983.
Bhattacharjee et al., J. Plant Bioch. and Biotech. 6, (2):69-73. 1997.
Bochkarev et al., Opin. Struct. Biol., 14:36-42, 2004.
Buchanan-Wollaston et al., 1992.
Buddelmeijer et al., J. Bacteriol., 191:161-168, 2009.
Buising et al., Mol Gen Genet, 243(1):71-81. 1994.
Callis et al., Genes Dev., 1:1183-1200, 1987.
Casa et al., Proc. Natl. Acad. Sci. USA, 90(23):11212-11216, 1993.
Chandler et al., The Plant Cell, 1:1175-1183, 1989.
Christou, et al., Proc. Natl. Acad. Sci. USA, 84:3962-3966, 1987.
Cinier et al., Bioconjugate Chem., 20:2270-2277, 2009.
Conkling et al., Plant Physiol., 93:1203-1211, 1990.
D'Halluin et al., Plant Cell, 4(12):1495-1505, 1992.
Dellaporta et al., Chromosome Structure and Function: Impact of New Concepts, 18th Stadler Genetics Symposium, 11:263-282, 1988.
De Block et al., EMBO Journal, 6(9):2513-2518, 1987.
De Block et al., Plant Physiol., 91:694-701, 1989.
EP Application 154,204 1985.
EP Application 2161278
Ebert et al., Proc. Natl. Acad. Sci., 84:5745-5749, 1987.
Fraley et al., Bio/Technology, 3:629-635, 1985.
Fromm et al., Nature, 319:791-793, 1986.
Gallie et al., The Plant Cell, 1:301-311, 1989.
Gelvin et al., 1990.
Ghosh-Biswas et al., J. Biotechnol., 32(1):1-10, 1994.
Haseloff et al., Proc. Natl. Acad. Sci. USA, 94(6):2122-2127, 1997.
He et al., Plant Cell Reports, 14 (2-3):192-196, 1994.
Hiei et al., Plant. Mol. Biol., 35(1-2):205-218, 1997.
Hinchee et al., Bio/Technol., 6:915-922, 1988.
Hou et al., Plant Physiology, 111:166, 1996.
Hudspeth et al., Plant Mol. Biol., 12:579-589, 1989.
Ikuta et al., Bio/technol., 8:241-242, 1990.
Ishida et al., Nat. Biotechnol., 14(6):745-750, 1996.
Kaeppler et al., Plant Cell Reports, 9:415-418, 1990.
Kaeppler et al., Theor. Appl. Genet., 84(5-6):560-566, 1992.
Katz et al., J. Gen. Microbiol., 129:2703-2714, 1983.
Klee et al., Bio-Technology, 3(7):637-642, 1985.
Knittel et al., Plant Cell Reports, 14(2-3):81-86, 1994.
Krehenbrink et al., J. Mol. Biol., 383:1058-1068, 2008.
Kyte & Doolittle et al., J. Mol. Biol., 157:105-132, 1982.
Lawton et al., Plant Mol. Biol. 9:315-324, 1987.
Lazzeri, Methods Mol. Biol., 49:95-106, 1995.
Lee et al., Korean J. Genet., 11(2):65-72, 1989.
Lorz et al., Mol Gen Genet, 199:178-182, 1985.
Marcotte et al., Nature, 335:454, 1988.
McCabe et al., 1993.
McCormac et al., Mol. Biotechnol., 9:155-159, 1998.
McKersie et al., Plant Physiol., 103:1155-1163, 1993.
Mouratou et al., Proc. Natl. Acad. Sci. USA, 104:17983-17988, 2007.
Murakami et al., Mol. Gen. Genet., 205:42-50, 1986.
Murzin, EMBO J., 12:861-867, 1993.
Nagatani et al., Biotech. Tech., 11(7):471-473, 1997.
Odell et al., Nature, 313:810-812, 1985.
Ogawa et al., Sci. Rep., 13:42-48, 1973.
Omirulleh et al., Plant Mol. Biol., 21(3):415-428, 1993.
Ow et al., Science, 234:856-859, 1986.
PCT Application WO 92/17598
PCT Application WO 94/09699
PCT Application WO 95/06128
PCT Application WO 97/4103
PCT Application WO 97/41228
PCT Application WO/2007/139397
PCT Application WO/2008/068637
PCT Application WO/WO2010/066740
Potrykus et al., Mol. Gen. Genet., 199:183-188, 1985.
Prasher et al., Biochem. Biophys. Res. Commun., 126:1259-1268, 1985.
Reichel et al., Proc. Natl. Acad. Sci., 93:5888-5893, 1996.
Rhodes et al., Methods Mol. Biol., 55:121-131, 1995.
Ritala et al., Plant Mol. Biol., 24:317-325, 1994.
Rogers et al., Methods Enzymol., 153:253-277, 1987.
Sambrook et al., In: Molecular Cloning—A Laboratory Manual, 1989.
Shakirov et al., Mol. Cell Biol. 25:7725-7733, 2005.
Sheen et al., Plant Journal, 8:777-784, 1995.
Singsit et al., Transgenic Res., 6:169-176, 1997.
Stalker et al., Science, 242:419-422, 1988.
Sullivan et al., Mol. Gen. Genet., 215:431-440, 1989.
Sutcliffe, Proc. Natl. Acad. Sci., 75:3737-3741, 1978.
Theobald et al., Annu. Rev. Biophys. Biomol. Struct., 32:115-133, 2003.
Thillet et al., J. Biol. Chem., 263:12500-12508, 1988.
Thomas et al., Plant Sci. 69:189-198, 1990.
Thompson et al., Euphytica, 85:75-80, 1995.
Thompson et al., The EMBO Journal, 6:2519-2523, 1987.
Tian, Plant Cell Rep., 16:267-271, 1997.
Tingay et al., The Plant Journal, 11:1369-1376. 1997.
Tomes et al., Plant. Mol. Biol., 14:261-268, 1990.
Toriyama et al., Theor Appl. Genet., 73:16, 1986.
Tsukada et al., Plant Cell Physiol., 30:599-604, 1989.
Twell et al., Plant Physiol.; 91:1270-1274, 1989.
Uchimiya et al., Mol. Gen. Genet., 204:204, 1986.
VanEck et al., 1995.
Vasil et al., Plant Physiol., 91:1575-1579, 1989.
Walder et al., Gene, 42:133-139, 1986.
Walker et al., Proc. Natl. Acad. Sci. USA, 84:6624-6628, 1987.
Wang et al., Molecular and Cellular Biology, 12:3399-3406, 1992.
Yamada et al., Plant Cell Rep., 4:85, 1986.
Yang et al., Proc. Natl. Acad. Sci. USA, 87:4144-4148, 1990.
Zheng & Edwards, J. Gen. Virol., 71:1865-1868, 1990.
Zhou et al., Plant Cell Reports, 12:612-616, 1993.
Zukowsky et al., Proc. Natl. Acad. Sci., 80:1101-1105, 1983.

1. A method for binding a target molecule comprising expressing a recombinant polypeptide which forms an alpha-helical coiled-coil structure in a plant and contacting the target molecule with the polypeptide, wherein the polypeptide forms an alphabody protein, wherein the target molecule is a protein or a carbohydrate that is present in cells of the midgut of the insect pest.

2-5. (canceled)

6. The method of claim 1, wherein the polypeptide which forms an alpha-helical coiled-coil structure comprises a sequence selected from the group consisting of SEQ ID NOs:20-25.

7. The method of claim 6, wherein the expressed polypeptide further comprises a localization sequence.

8. The method of claim 1, wherein the polypeptide which forms an alpha-helical coiled-coil structure comprises 1-3 ligand binding domains.

9. The method of claim 1, wherein contacting the target molecule with the polypeptide which forms an alpha-helical coiled-coil structure comprises contacting the plant with the target molecule; or wherein contacting the target molecule with the polypeptide which forms an alpha-helical coiled-coil structure comprises providing the polypeptide which forms an alpha-helical coiled-coil structure in the diet of an insect pest wherein the insect pest comprises the target molecule.

10. (canceled)

11. The method of claim 1, wherein the polypeptide which forms an alpha-helical coiled-coil structure is produced by a method comprising:
(a) producing a plurality of recombinant coding sequence for a polypeptide which forms an alpha-helical coiled-coil structure to generate a library of recombinant protein sequences; and
(b) screening the library of recombinant sequences to identify a polypeptide which forms an alpha-helical coiled-coil structure that binds to a target molecule;
or wherein the method comprises:
(a) producing a plurality of recombinant coding sequences for a polypeptide which forms an alpha-helical coiled-coil structure to generate a library of recombinant sequences which encode a polypeptide which forms an alpha-helical coiled-coil structure;
(b) screening the library of sequences which encode a polypeptide which forms an alpha-helical coiled-coil structure to identify a recombinant polypeptide sequence that binds to a target molecule;
(c) producing a further plurality of recombinant coding sequences for the polypeptide which forms an alpha-helical coiled-coil structure to generate a further library of coding sequences; and
(d) screening the further library of coding sequences to identify a sequence of a polypeptide which forms an alpha-helical coiled-coil structure, with enhanced binding to a target molecule;
or wherein the method further comprises, after steps (a)-(d):
(e) repeating steps (c)-(d) one or more times to generate a polypeptide which forms an alpha-helical coiled-coil structure with further enhanced binding to a target molecule.

12. The method of claim 11, wherein screening the library comprises the use of phage display or bacterial anchored periplasmic expression to identify binding of a polypeptide which forms an alpha-helical coiled-coil structure, to a target molecule; or wherein the binding of a polypeptide which forms an alpha-helical coiled-coil structure, to a target molecule is identified by fluorescence-activated cell sorting or magnetic separation.

13. The method of claim 1, wherein the polypeptide which forms an alpha-helical coiled-coil structure, is produced by a method comprising:
(a) producing a plurality of polypeptides which form an alpha-helical coiled-coil structure; and
(b) screening the polypeptides which form an alpha-helical coiled-coil structure, to identify a polypeptide which forms an alpha-helical coiled-coil structure, that binds to a target molecule.

14. The method of claim 13, wherein identifying a polypeptide which forms an alpha-helical coiled-coil structure, that binds to a target molecule comprises determining the sequence of a polypeptide which forms an alpha-helical coiled-coil structure;
optionally wherein the sequence is determined by mass spectroscopy.

15. (canceled)

16. The method of claim 1, wherein the target molecule is an ACCase inhibitor, ALS inhibitor, a triazine, a bypyridilium, an auxin, glycine or a triketone; wherein the protein target molecule is a protein expressed in the plant; wherein the protein target molecule is a peritrophin, chitinase, aminopeptidase, cystein protease, trypsin, chymotrypsin or carboxypeptidase; wherein the carbohydrate target molecule is chitin; wherein the target molecule is present in the gut of an insect pest; or wherein the binding of the target molecule occurs in the plant.

17. The method of claim 1, wherein expressing the polypeptide which forms an alpha-helical coiled-coil structure in the plant confers a trait of agronomic interest to the plant.

18. The method of claim 17, wherein the trait of agronomic interest is selected from the group consisting of herbicide tolerance, drought resistance, insect resistance, fungus resistance, virus resistance, bacteria resistance, male sterility, cold tolerance, salt tolerance, increased yield, enhanced oil composition, increased oil content, enhanced nutrient use efficiency and altered amino acid content.

19. The method of claim 1, wherein the polypeptide which forms an alpha-helical coiled-coil structure binds to at least two different target molecules; or wherein the sequence encoding the polypeptide which forms an alpha-helical coiled-coil structure is fused to a second coding sequence encoding a polypeptide of agronomic interest.

20. The method of claim 19, wherein the second coding sequence encodes a polypeptide which forms an alpha-helical coiled-coil structure; or wherein the second coding sequence encodes a pore-forming toxin.

21. The method of claim 1, wherein the plant is selected from the group consisting of wheat, maize, rye, rice, corn, oat, barley, turfgrass, sorghum, millet, sugarcane, tobacco, tomato, potato, soybean, cotton, canola, sunflower and alfalfa.

22-32. (canceled)
